(19) United States
(12) Patent Application Publication
(10) Pub. No.: US 2008/0299141 A1
(43) Pub. Date: Dec. 4, 2008

(54) Raccoon Poxvirus Expressing Genes of Porcine Virus

(75) Inventors: Stephen Qitu Wu, Fort Dodge, IA (US); Michael A. Gill, Fort Dodge, IA (US); Hsien-Jue Chu, Bonner Springs, KS (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/128,240

(22) Filed: May 28, 2008

Related U.S. Application Data
(60) Provisional application No. 60/932,421, filed on May 30, 2007.

(51) Int. Cl. A61K 39/00; C12N 15/00; A61P 37/00

(52) U.S. Cl. 424/186.1; 435/320.1

(57) ABSTRACT

The present invention relates to a new recombinant raccoon poxvirus vector vaccine in which the vector expresses one or more antigenic proteins encoded by multiple open reading frames, preferably the ORF5, ORF6 and/or ORF3/ORF4/ORF7, of one or more porcine reproductive and respiratory syndrome virus strains alone or in combination with an open reading frame of porcine circovirus type 2 (PCV-2), preferably ORF2, at the hemagglutinin (ha) and/or thymidine kinase (tk) loci.

### CROSS-REFERENCE TO RELATED APPLICATIONS

This application claims the benefit under 35 U.S.C. §119 (e) of U. S. Provisional Application Ser. No. 60/932421, filed May 30, 2007, the disclosure of which is incorporated by reference herein in its entirety.

### FIELD OF THE INVENTION

The present invention concerns a new recombinant raccoon poxvirus vector that expresses the genes of porcine reproductive and respiratory syndrome virus (PRRSV) alone or in combination with porcine circovirus (PCV) and its use as a vaccine in the prophylaxis of disease caused by PCV and/or PRRSV.

### BACKGROUND OF THE INVENTION

Porcine reproductive and respiratory syndrome virus (PRRSV) is a positive-strand RNA virus, a member of the family Arteriviridae, genus Arterivirus. PRRSV infects swine, causing “Mystery Swine Disease” that was initially detected in 1987 in North America, then the disease spread to and through Europe in 1990. Since 1990, PRRSV has become a major global threat to the swine industry, affecting heavy production losses in pig herds.

Now known as Porcine Reproductive and Respiratory Syndrome (PRRS), the Mystery Swine Disease (also called Blue Ear disease, Porcine Epidemic Abortion and Respiratory Syndrome (PEARS) or Respiratory Syndrome (SIRS)) is characterized by reproductive failure of pregnant sows that results in an abnormally high number of abortions during the last phase of gestation, anorexia, stillbirths, mummified fetuses, post-farrowing respiratory problems and weak piglets that die shortly after birth of respiratory disease or secondary infections. In piglets, infection with the PRRSV causes respiratory distress and a high mortality rate that can reach as high as 70% mortality in piglets and greater. Older pigs may show mild signs of respiratory disease or secondary infections. The virus spreads quickly in the swine populations, and up to 95% of swine in a herd can become sero-positive within two to three months after initial infection. Even if the infections become stabilized, there is a strong likelihood that the PRRSV shed by excreting young female swine will infect the pregnant sows and the reproductive problems will reoccur. The economic impact of PRRS in pig breeding herds is considerable.

Isolated first in the Netherlands as the cause of the Mystery Swine Disease, PRRSV was identified as Lelystad virus (LV) (G. Wesvoort et al., Vet. Quarterly 3:121-130 (1991)). Shortly thereafter, a different Olot isolate was found in Spain (Plana et al., Vet. Microbiol., 33:203.211, 1992). Subsequently, new isolates of PRRSV have been described in several publications (see, for example, EP application No. 0 529 584 A2, WO 93/06211 and WO 93/07898).

Further publications revealed the structural characteristics of the PRRS virus isolates such as the entire Lelystad virus (LV) genome (J. J. M. Meulenberg et al., “Lelystad virus, the causative agent of porcine epidemic abortion and respiratory syndrome (PEARS), is related to LDV and EAV,” Virology 192: 62-72 (1993)); a segment of the Tubingen (Germany) PRRS virus isolate (TV) genome (K-K. Cozelmann et al., “Molecular characterization of porcine reproductive and respiratory syndrome virus, a member of the Arterivirus group,” Virology 193: 329-339 (1993)) and other isolates, permitting the genes to be cloned and sequenced.

Arterivirus virions, in general, are 50-70 nm in diameter and contain an isomeric (probably icosahedral) nucleocapsid, 35 nm in diameter, surrounded by a closely adherent envelope with honeycomb-like surface structures. The genome consists of a single molecule of linear positive-sense, ssRNA, 13 to 15 kb in size. The PRRS virus has a size of 50-60 nm, with an envelope of approximately 30-35 nm contained in the nucleocapsid, and a single RNA molecule. The genomic RNA of PRRSV is approximately 15 kb (15000 base pairs). The genome encodes the RNA replicase (ORF1a and ORF1b), the glycoproteins GP2 to GP5, the integral membrane protein M, and the nucleocapsid protein N (ORFs 2 to 7). Genomic RNA is synthesized via full-length, negative-sense replicative intermediate.

The single strand RNA molecule of the PRRSV genome contains a poly-A tail at 3′ end. The structure contains seven open reading frames (ORFs) encoding the viral proteins. ORF1 to ORF7 show small overlapping segments between them. Synthesis of the viral proteins is produced from a group of different length subgenomic transcripts (mRNA), but of similar 3′ polyadenylated end, and 5′ leader sequence originating from the non-coding 5′ end sequence. This form of viral protein expression is considered nested mRNAs and has been previously described for coronaviruses (W. J. M. Spaan et al., J. Gen. Virol. 69:2939-2952 (1988)). Based on the Lelystad (LV) and Tubingen (TV) PRRSV viral isolate nucleotide sequence, and by homology with what has been observed with other arterivirusesit has been proposed that in the viral genome, ORF1 (a and b) code for viral polymerase and replicase. ORF2 to ORF6 encode the viral envelope proteins, and ORF7 encode the nucleocapsid protein. Viral replicase and polymerase are large-sized proteins, 260 and 163 kDa respectively, and both of them contain three possible glycosylation sites. Envelope proteins (ORFs 2 to 6) located at 3′ end are small, between 30 and 19 kDa. All of them contain more than two possible glycosylation sites, especially ORF3 which contains 7 sites. All of these proteins contain hydrophobic sequences at the amino (N-) and carboxy (C-) terminal ends that might work as leader sequence and membrane anchor. Generally, they are hydrophobic proteins, in accord with their location associated to a membrane. ORF6 has 3 hydrophobic segments located within the 90 amino acid residues at the N-terminal end. The protein coded by ORF7, possibly corresponding to the viral nucleocapsid, is extremely basic with arginine, lysine and histidine residues at the N-terminal end. The amino acid sequences of LV and TV viral polymerase, structural proteins and nucleocapsid show an identity of between 29% and 67% in comparison with the LDV virus, and between 20% and 36% in comparison with the EAV virus.

The disease caused by PRRSV is responsible for severe losses to the global pig industry. For this reason, research has been spent developing vaccines capable of preventing the infection caused by PRRSV. The basis for the older vaccine development is the fact that PRRSV grows in primary alveolar macrophages of the host cells (the pig) and in monkey kidney cell lines. In large part, the prior vaccines against PRRSV, which have been described in WO 92/21375, WO 93/06211, WO 93/07898 and ES P9301973, are conventional vaccines obtained from viruses grown on macrophages and subsequently inactivated. The vaccines are disclosed as being capable of avoiding Porcine Reproductive and Respiratory Syndrome (PRRS) and preventing reproductive alterations in sows.

Also of major significance to the welfare of pigs is another wide-spread disease called Postweaning Multisystemic Wasting Syndrome (PMWS). PMWS, which typically affects weaning piglets between around 5-18 weeks of age, poses a current or potential health threat to the piglets in epidemic proportions and seriously impacts the economic interests of the global swine industry. Clinical signs of PMWS consist of progressive weight loss, dyspnea, tachypnea, anemia, diarrhea and jaundice. Although more prevalent in weaning piglets, the debilitating disease can affect any animal of the porcine family including swine, hogs and adult pigs. Mortality rate may vary from 1% to 2%, and up to 40% in some complicated cases throughout the world. Porcine circovirus type 2 (commonly referred to as PCV-2) has been identified as the primary causative agent of PMWS (G. M. Allan et al., “Novel porcine circoviruses from pigs with wasting disease syndromes,” Vet. Rec., Vol. 142, pages 467-468, 1998; G. M. Allan et al., “Isolation of porcine circovirus-like viruses from pigs with a wasting disease in the USA and Europe,” J. Vet. Diagn. Invest., Vol. 10, pages 3-10, 1998). It is a critical goal within the commercial swine business, therefore, to prevent outbreaks of PMWS in the pig family through the development of an effective vaccine against PCV2, the etiologic agent of PMWS, as a target for mass vaccination of the animals at risk.

U.S. Pat. No. 6,217,883, corresponding to French Patent No. 2,781,159B, concerns five isolated PCV strains from pulmonary or ganglionic samples taken from pigs infected with PMWS in Canada, California and France and their use in combination with at least one porcine parvovirus antigen in multivalent immunogenic compositions. Proteins encoded by PCV2 open reading frames (ORF) consisting of ORF1 to ORF13 are broadly described in the patent but there is no exemplification of any specific protein exhibiting immunogenic properties. The patent discloses vectors consisting of DNA plasmids, linear DNA molecules and recombinant viruses that contain and express in vivo a nucleic acid molecule encoding the PCV antigen.

U.S. Pat. No. 6,368,601 relates to new porcine circovirus strains isolated from pulmonary or ganglionic samples obtained from farms affected by the post-weaning multisystemic wasting syndrome (PMWS). In particular, the patent describes purified preparations of these strains, conventional attenuated or inactivated vaccines, recombinant live vaccines, plasmid vaccines and subunit vaccines, as well as reagents and diagnostic methods. The patent further discloses a vector comprising isolated DNA molecule sequences which can be used for the production of subunits in an in vitro expression vector or as sequences to be integrated into a virus or plasmid type in vivo expression vector. The patent broadly describes their vector as being a pig herpes virus, a porcine adenovirus or a poxvirus and, more particularly, Aujesky's disease virus, vaccinia virus, avipox virus or swinepox virus.

Previously, certain porcine reproductive and respiratory syndrome virus (PRRSV) genes (ORF2, ORF3, ORF4, ORF5, ORF6, ORF7) have been expressed in different vector systems: Baculovirus, Psuedorabies virus, TGEV and plasmid DNA. All of these expression systems are limited by being capable of expressing only one or two open reading frames in one viral construct.

For instance, U.S. Pat. No. 5,888,513 relates to the recombinant proteins of the causative virus of Porcine Reproductive and Respiratory Syndrome (PRRS), corresponding to ORF2 to ORF7 of the PRRSV Spanish isolate (PRRS-Olot), that have been produced in baculovirus expression system using Sf 9 (Spodoptera frugiperda insect cells) cell cultures as a permissive host. The patent describes the recombinant proteins as suitable for the formulation of vaccines capable of protecting porcine livestock from PRRS and for the preparation of diagnostic kits for detection of anti-PRRSV antibodies as well as of PRRSV in a pig biological sample. Specifically, the patent concerns a recombinant expression system comprising a recombinant baculovirus and a method of expressing isolated viral subunit proteins from at least one isolated nucleotide sequence selected from the group consisting of ORF2 to ORF7 of the PRRS-Olot isolate from a baculovirus transfer vector. The patent also describes a recombinant vaccine that contains at least one recombinant PRRSV protein.

There is a significant amount of published information on the topic of recombinant raccoon poxvirus for the expression of porcine antigens and use as vaccines. For instance, U.S. Pat. No. 6,294,176 discloses a recombinant raccoon poxvirus (RCNV) vector that provides the insertion of one or two foreign DNA sequences of the open reading frames (ORFs) of porcine reproductive and respiratory syndrome virus (PRRSV), swine influenza virus hemagglutinin (SIV HA), swine influenza virus neuraminidase (SIV NA) and porcine parvovirus (PPV), such as one of ORF2-7 of PRRSV, in a non-essential region within the HindIII “U” genomic region, the HindIII “M” genomic region or the HindIII “N” genomic region of the raccoon poxvirus genome. The raccoon poxvirus viral genome is described in the patent as containing a deletion in the raccoon poxvirus host range gene of the viral genome. The patent provides a homology vector for producing the recombinant raccoon poxvirus by inserting the foreign DNA sequence into the raccoon poxvirus genome. In the examples, the patent only shows how to prepare the insertion of the foreign DNA into the HindIII “U” genomic region. DNA sequence analysis indicates that the HindIII “U” genomic region disclosed by the patentees is not the hemagglutinin (ha) insertion and/or the thymidine kinase (tk) regions of the recombinant raccoon poxvirus genome.

U.S. Pat. No. 7,109,025 discloses viral vectors and vaccines using recombinant porcine adenoviruses. In particular, the patent describes an in vivo replicating and recombinant porcine adenovirus comprising a heterologous nucleotide sequence inserted into the porcine adenovirus under conditions enabling the latter to be replicated in vivo and to express the inserted heterologous nucleotide sequence. The patent further describes using an adenovirus genome from porcine adenovirus (PAV) serotype 3 or 5 (PAV-3 or PAV-5) and inserting the heterologous nucleotide sequence encoding at least one product in a non-essential zone of the E3 region of the PAV-3 or PAV-5 genome located downstream of the major late promoter (MLP). The patent also relates to a DNA fragment comprising all or part of the referenced nucleotide sequence. The recombinant PAV vector is shown to express a porcine pathogenic agent selected from the group consisting of pseudorabies virus, swine influenza virus, porcine reproductive and respiratory syndrome AM102713 virus, parvovirus virus, Hog Cholera Virus, porcine circovirus type 2, Actinobacillus pleuropneumoniae and Mycoplasma hyopneumoniae.

U.S. Pat. Number 6,497,883 deals with the use of a recombinant poxvirus, such as avipox virus, containing foreign DNA from porcine circovirus type 2 along with methods of treating or preventing disease caused by porcine circovirus type 2. Specifically, the recombinant poxvirus of the patent comprises an isolated DNA molecule consisting of ORFs 1 to 13 of porcine circovirus type 2, wherein the recombinant poxvirus is a recombinant ALVAC canarypox virus.

U.S. Pat. No. 7,211,379 concerns a method for reducing viral load of porcine circovirus type 2 (PCV-2) in a pig by inducing an immune response against PCV-2 through the administration of an immunogenic composition comprising a PCV-2 antigen. The patent describes the antigen as a vector containing an exogenous PCV-2 nucleotide sequence, wherein the PCV-2 nucleotide sequence encodes and expresses ORF4, ORF13, or ORF4 and ORF13. In some embodiments shown in the patent, the immunogenic composition includes one or more additional pig pathogens. The composition used in the method disclosed in the patent additionally comprises at least one immunogen from at least one additional pig pathogen or a vector expressing such an immunogen in which the additional pig pathogen is selected from the group consisting of PRRS, Mycoplasma hyopneumoniae, Actinobacillus pleuropneumoniae, Escherichia coli, atrophic rhinitis, pseudorabies, hog cholera, swine influenza, encephalomyocarditis virus, and PPV. The vectors disclosed in the patent comprise a DNA vector plasmid, a bacteria such as an E. coli, a virus such as baculovirus, a herpesvirus including pig herpes viruses or Aujeszky's disease virus (pseudorabies), an adenovirus including a porcine adenovirus, and a poxvirus including a vaccinia virus, an avipox virus, a canarypox virus, a racoonpox and a swinepox virus.

The disclosures of U.S. Pat. No. 6,241,989 and its continuation U.S. Pat. No. 7,087,234 that deal with multivalent recombinant raccoon poxviruses, containing more than one exogenous gene inserted into either the thymidine kinase gene or the hemagglutinin gene. Disclosed in these two related patents is the use of the multivalent recombinant raccoon poxviruses as vaccines to immunize felines against subsequent challenge by feline pathogens. Also disclosed is a method of making a multivalent recombinant raccoon poxvirus by a recombinant process involving the construction of an insertion vector into which the exogenous genes are inserted; and flanking the inserted genes are sequences which can recombine into the raccoon poxvirus thymidine kinase gene or the hemagglutinin gene; introducing both the insertion vector containing the exogenous genes, and raccoon poxvirus into susceptible host cells; and selecting the recombinant raccoon poxvirus from the resultant plaques. The multivalent, recombinant raccoon poxvirus of the patents can infect and replicate in feline cells, and contains more than one exogenous gene inserted into a region consisting of a hemagglutinin gene or a thymidine kinase gene of the raccoon poxvirus genome which is non-essential for viral replication, notably wherein the exogenous genes are operably linked to a promoter for expression; and each exogenous gene encodes a feline pathogen antigen. The patents describe exogenous genes encoding feline pathogen antigens such as feline leukemia virus (FeLV Env), feline immunodeficiency virus (FIV Gag), feline immunodeficiency virus (FIV Env), feline infectious peritonitis virus (FIPV M), feline infectious peritonitis virus (FIPV N), feline calicivirus (FCV capsid protein), feline panleukopenia virus (FPV VP2) and rabies-G.

Despite all the efforts made in the veterinary vaccine art, there is still an art-recognized need for a safe and effective monovalent or multivalent PCV and/or PRRSV recombinant vaccine that would adequately protect pigs from being infected with the virus or viruses. Also needed is a safe and effective method for the prevention of infection or disease caused by PCV and/or PRRSV through the administration of a recombinant vaccine that can give an adequate protective immune response in a pig against PMWS and/or PRRS. In addition to safety and efficacy upon administration to pigs, other benefits over the conventional, inactivated PRRSV vaccines would be attained by the recombinant vaccine that eliminates the risk of an accidental infection from the free virus or the inactivated virus reverting to its pathogenic state. It is clear that the development of a safe and effective raccoon poxvirus-vectored porcine vaccine would result in important advantages in usage to prevent serious pig diseases, to ensure employee safety during vaccine production and to completely eliminate any chance of the pathogenic viruses surviving inactivation and decontamination procedures used in commercial production.

All patents and publications cited in this specification are hereby incorporated by reference in their entirety.

### SUMMARY OF THE INVENTION

In its broadest aspect, the invention provides a safe and effective recombinant poxvirus vector vaccine in which the vector expresses one or more of the antigenic proteins encoded by one or more porcine reproductive and respiratory syndrome virus (PRRSV) isolates, preferably the antigenic proteins encoded by the multiple open reading frames of one or more porcine reproductive and respiratory syndrome virus (PRRSV) isolates, alone or in combination with one or more open reading frames of porcine circovirus type 2 (PCV-2) at the ha and/or tk loci. In a particular embodiment, the vector further comprises a nucleic acid molecule encoding the glycoprotein of the feline leukemia virus bearing the FeLV P27⁺ phenotype. The FeLV P27⁺ or PCV-2 capsid gene is useful as a unique marker to screen the clones in different combinations. This invention also provides a method of protecting pigs against Postweaning Multisystemic Wasting Syndrome and/or Porcine Reproductive and Respiratory Syndrome by administering the potent new recombinant vaccine to pigs in need of protection.

Accordingly, a first aspect of the invention provides a recombinant raccoon poxvirus vector (rRCNV) comprising one or more exogenous nucleic acid molecules encoding a porcine reproductive and respiratory syndrome virus (PRRSV) protein, wherein:
a.) at least one nucleic acid molecule is inserted into the hemagglutinin locus or the thymidine kinase locus of the raccoon poxvirus genome; or
b.) at least two nucleic acid molecules are inserted into the hemagglutinin locus or the thymidine kinase locus of the raccoon poxvirus genome; or
c.) at least one nucleic acid molecule is inserted into the hemagglutinin locus and at least one nucleic acid molecule is inserted into the thymidine kinase locus of the raccoon poxvirus genome; or

In one embodiment, the raccoon poxvirus vector comprises at least two nucleic acid molecules that are inserted into the hemagglutinin locus and at least two nucleic acid molecules that are inserted into the thymidine kinase locus of the raccoon poxvirus genome.

In one embodiment, the recombinant raccoon poxvirus vector further comprises a nucleic acid molecule encoding a porcine reproductive and respiratory syndrome virus (PRRSV) protein that is inserted into a third non-essential site of the raccoon poxvirus genome in addition to the thymidine kinase and the hemagglutinin loci of the raccoon poxvirus genome. Any non-essential site of the recombinant raccoon poxvirus genome may be contemplated for use in the invention.

In one embodiment, the third non-essential site of the raccoon poxvirus genome is the serine protease inhibitor site.

In one embodiment, each of the one or more exogenous nucleic acid molecules encodes one or more open reading frames of a porcine reproductive and respiratory syndrome virus. The open reading frames are selected from the group consisting of ORF1, ORF2, ORF3, ORF4, ORF5, ORF6, ORF7 and a combination thereof.

In one embodiment, the poxvirus vector encodes at least two copies of the same porcine reproductive and respiratory syndrome virus protein. The two copies of the same protein may be encoded by two different nucleic acid molecules, or they may be encoded by the same nucleic acid molecule. The two copies of the same protein may be operably linked to one promoter, or the two copies of the same protein may be operably linked to separate promoters. The two copies of the same protein are encoded by a porcine reproductive and respiratory syndrome virus open reading frame selected from the group consisting of ORF1, ORF2, ORF3, ORF4, ORF5, ORF6, ORF7 and a combination thereof.

In one embodiment, the two or more nucleic molecules are inserted into the hemagglutinin locus and/or the thymidine kinase locus of the raccoon poxvirus genome.

In one embodiment, the two or more exogenous nucleic acid molecules encode at least two open reading frames of porcine reproductive and respiratory syndrome virus selected from the group consisting of ORF5-ORF6, ORF5-ORF6-ORF7, ORF3-ORF5-ORF6, ORF3-ORF4-ORF7, ORF3-ORF4-ORF5-ORF7, ORF3-ORF4-ORF6-ORF7, and ORF3-ORF4-ORF5-ORF6-ORF7.

In one embodiment, the porcine reproductive and respiratory syndrome virus is ISU12 (Iowa), Olot/91 (Spanish) or both.

In one embodiment, the recombinant raccoon poxvirus vector further comprises a nucleic acid molecule encoding an open reading frame (ORF) of porcine circovirus type 2 (PCV2).

In one embodiment, the porcine circovirus 2 open reading frame is ORF2.

In one embodiment, the PCV2 ORF2 may be separately inserted into the thymidine kinase locus of the raccoon poxvirus genome.

In one embodiment, the recombinant raccoon poxvirus vector further comprises a nucleotide sequence encoding a glycoprotein of feline leukemia virus P27⁺ phenotype.

In one embodiment, the raccoon poxvirus is live and replicable.

A second aspect of the invention provides a recombinant porcine vaccine comprising an immunologically effective amount of any one or more of the recombinant raccoon poxvirus vectors described herein, and, optionally, a suitable carrier, diluent or adjuvant.

In one embodiment, the recombinant porcine vaccine comprises an immunologically effective amount of two or more of the recombinant raccoon poxvirus vectors as described herein, and, optionally, a suitable carrier or diluent.

In one embodiment, the raccoon poxvirus is live and replicable.

In one embodiment, the vaccine is administered as a single dose or as repeated doses.

In one embodiment, the recombinant porcine vaccine further comprises at least one additional porcine antigen. The additional porcine antigen is selected from the group consisting of Mycoplasma hyopneumoniae antigen, Haemophilus parasuis antigen, Pasteurella multocida antigen, Streptococcum suis antigen, Actinobacillus pleuropneumoniae antigen, Bordetella bronchiseptica antigen, Salmonella choleraesuis antigen, Erysipelothrix rhusiopathiae antigen, leptospira bacteria antigen, swine influenza virus antigen, porcine parvovirus antigen, Escherichia coli antigen, porcine respiratory coronavirus antigen, rotavirus antigen, an antigen from a pathogen causative of Aujesky's Disease, Swine Transmissible Gastroenteritis antigen and a combination thereof.

A third aspect of the invention provides a method for inducing an immune response against porcine reproductive and respiratory syndrome virus, comprising administering to a porcine animal a recombinant porcine vaccine comprised of one or more of the recombinant raccoon poxvirus vectors as described herein.

In one embodiment, the immune response is a humoral or antibody mediated response.

In one embodiment, the immune response is a cell-mediated or T cell mediated immune response.

In one embodiment, the protective immune response is induced by administering a vaccine dose that ranges from about 4.5 Log₁₀TCID₅₀/ml to about 7.5 Log₁₀/TCID₅₀/ml.

In another embodiment, the recombinant porcine vaccine utilized for inducing an immune response comprises one or more of a live and replicable recombinant raccoon poxvirus vector(s).

In another embodiment, the recombinant porcine vaccine utilized for inducing an immune response comprises two or more live and replicable recombinant raccoon poxvirus vectors.

A fourth aspect of the invention provides a method for preventing porcine reproductive and respiratory syndrome and Postweaning Multisystemic Wasting Syndrome, comprising administering to a porcine animal in need of protection a recombinant porcine vaccine comprising an immunologically effective amount of one or more of the recombinant raccoon poxvirus vector(s) as described herein, and optionally, a suitable carrier, diluent or adjuvant.

In one embodiment, the recombinant raccoon poxvirus vector(s) is/are live and replicable.

### BRIEF DESCRIPTION OF THE DRAWINGS

FIG. 1 is the nucleic acid sequence of the pFD2000A-FDAH plasmid (SEQ ID NO: 1)

FIG. 2 is the nucleic acid sequence of the pFD2001 TK-FDAH plasmid (SEQ ID NO: 2)

FIG. 3 is the nucleic acid sequence of the pFD2003SEL-FDAH plasmid (SEQ ID NO: 3)

FIG. 4 is the nucleic acid sequence of the pFD2003SEL-GPV-PV-FDAH plasmid (SEQ ID NO: 4)

FIG. 5 is the nucleic acid sequence of PCV2A ORF2-FDAH (SEQ ID NO: 5)

FIG. 6 is the nucleic acid sequence of PCV2B ORF2-FDAH (SEQ ID NO: 6)

FIG. 7 is the nucleic acid sequence of PRRSV ISU12-ORF3-FDAH (SEQ ID NO: 7)

FIG. 8 is the nucleic acid sequence of PRRSV ISU12-ORF4-FDAH (SEQ ID NO: 8)

FIG. 9 is the nucleic acid sequence of PRRSV ISU12-ORF5-FDAH (SEQ ID NO: 9)

FIG. 10 is the nucleic acid sequence of PRRSV ISU12-ORF6-FDAH (SEQ ID NO: 10)

FIG. 11 is the nucleic acid sequence of PRRSV ISU12-ORF7-FDAH (SEQ ID NO: 11)

FIG. 12 is the nucleic acid sequence of PRRSV Olot91-ORF5-FDAH (SEQ ID NO: 12)

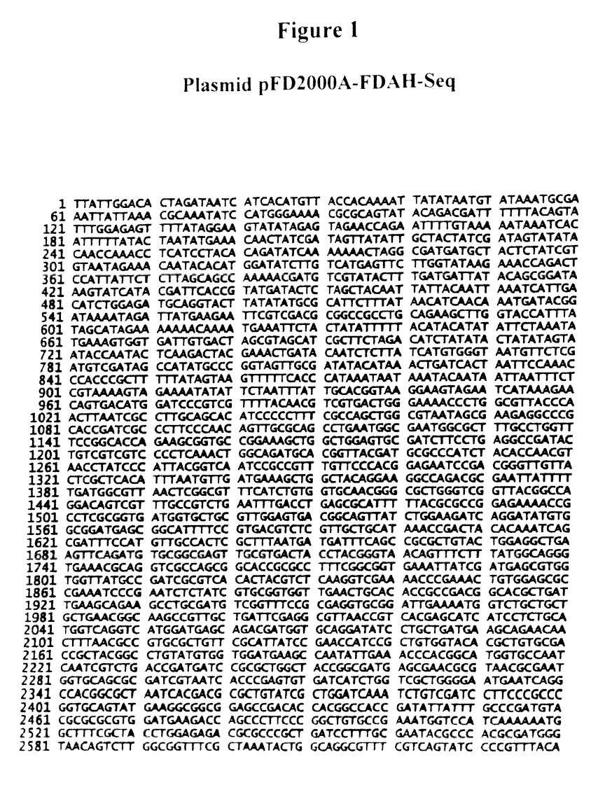

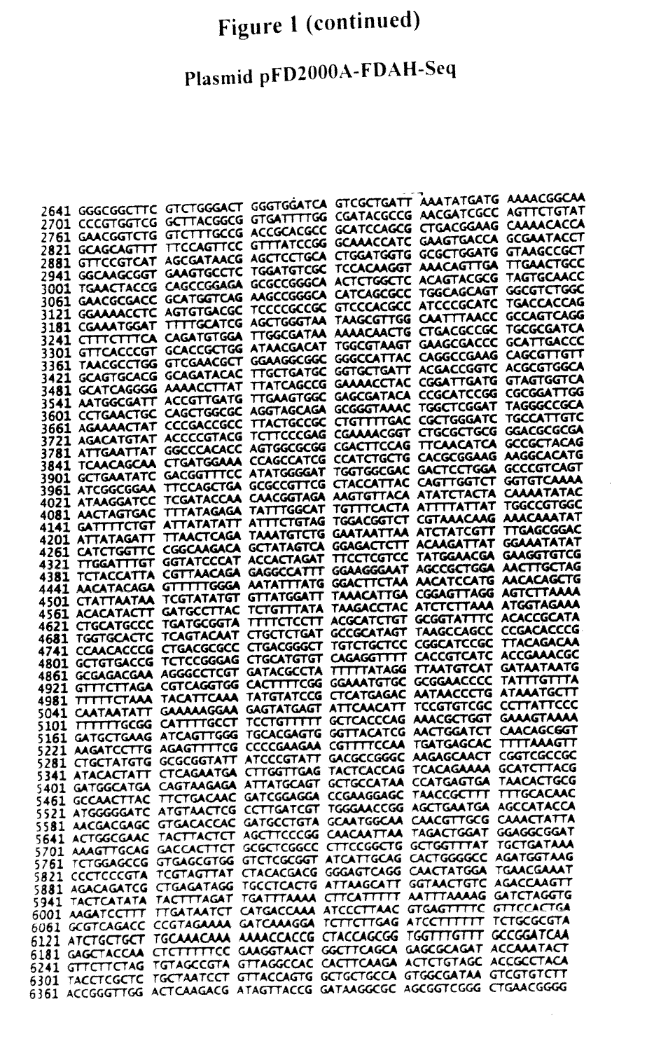

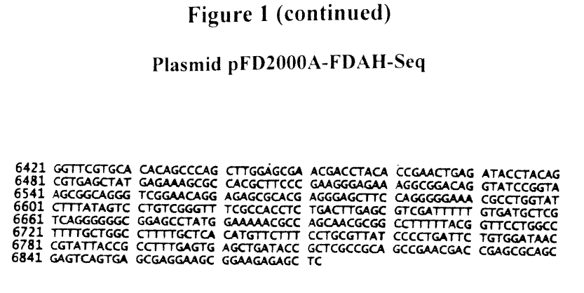

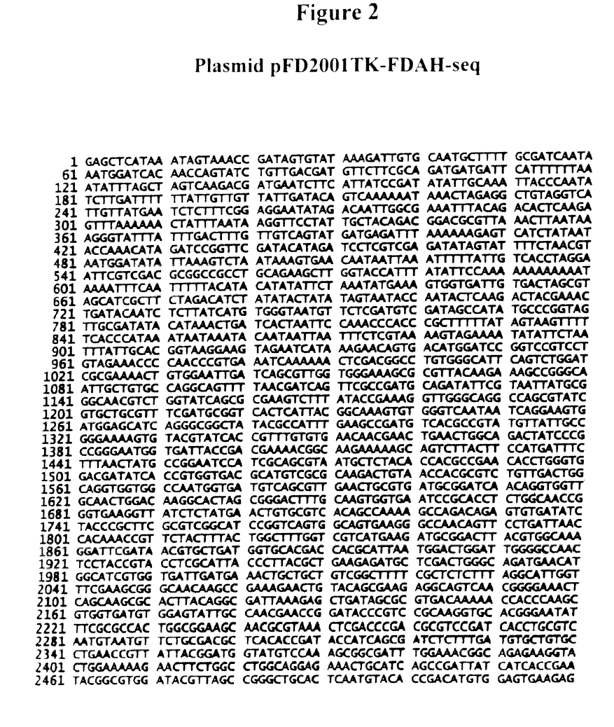

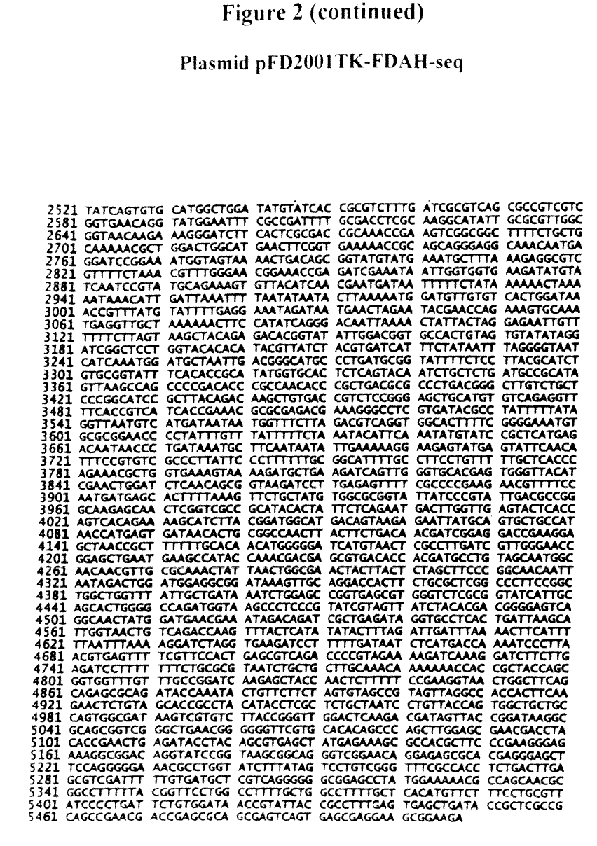

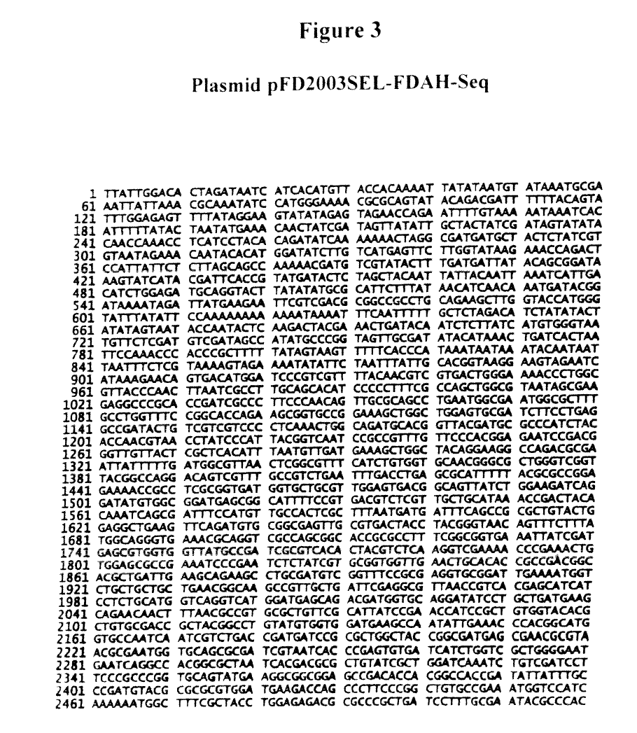

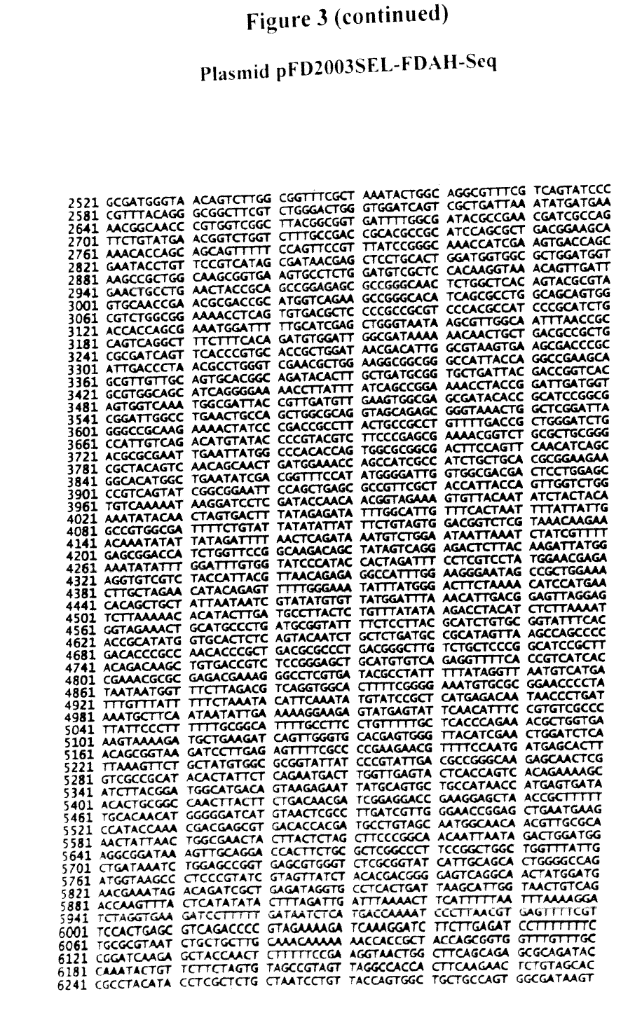

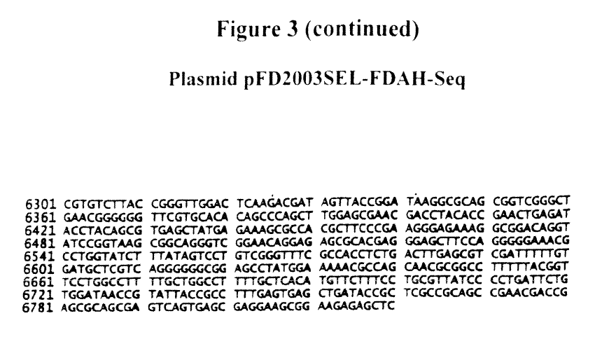

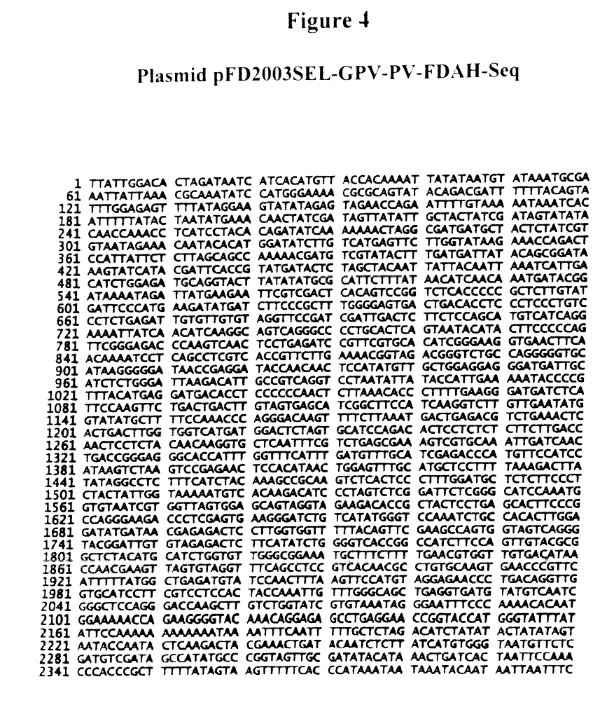

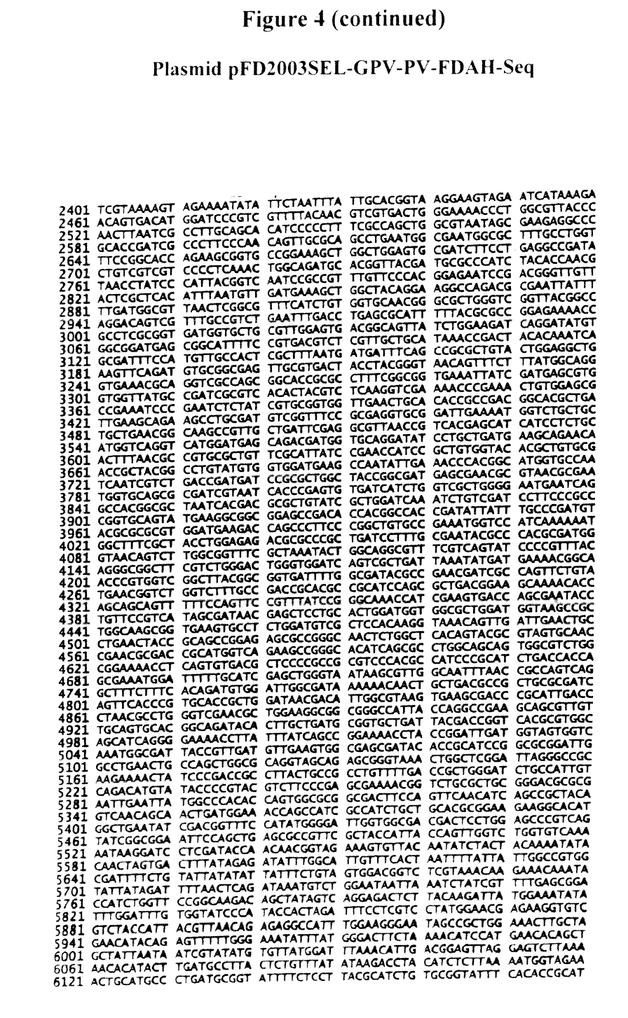

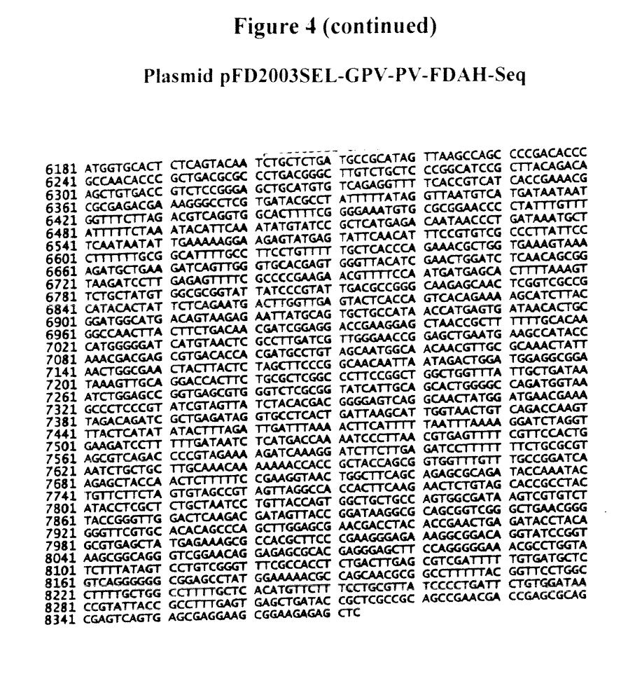

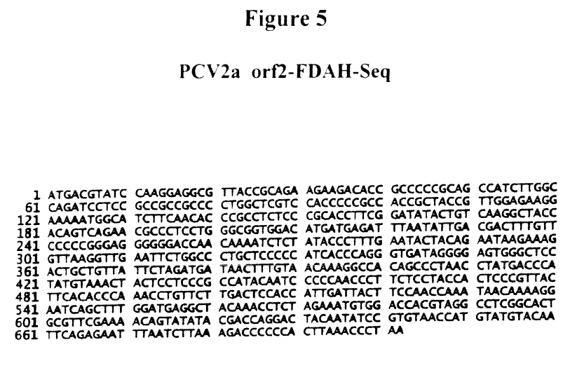

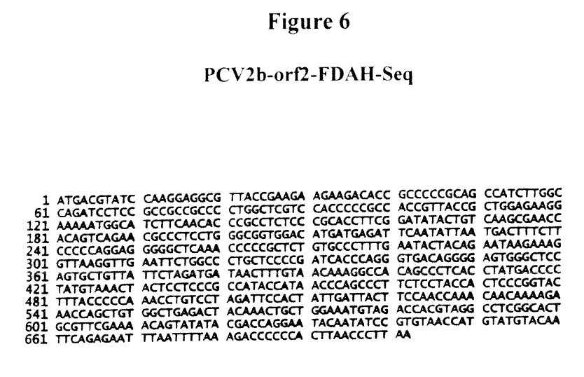

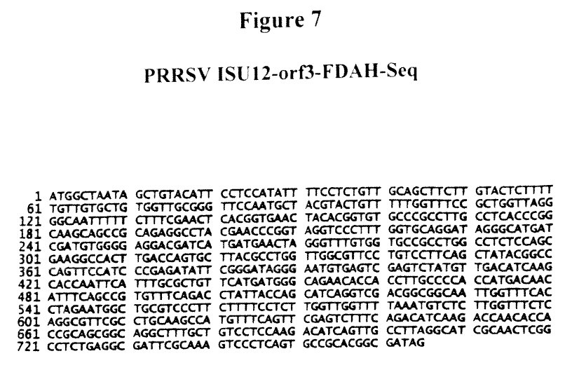

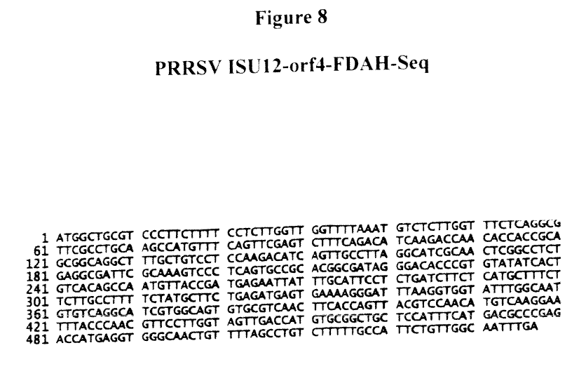

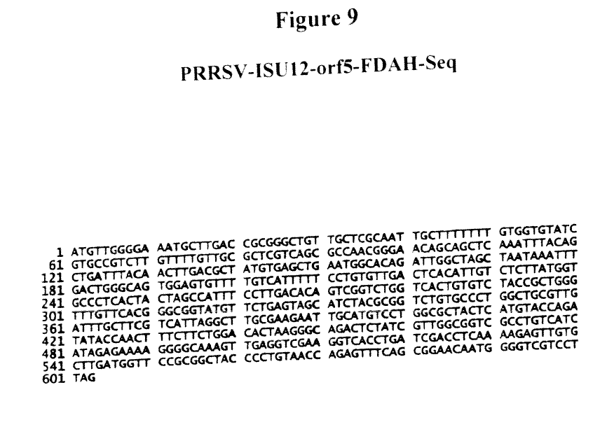

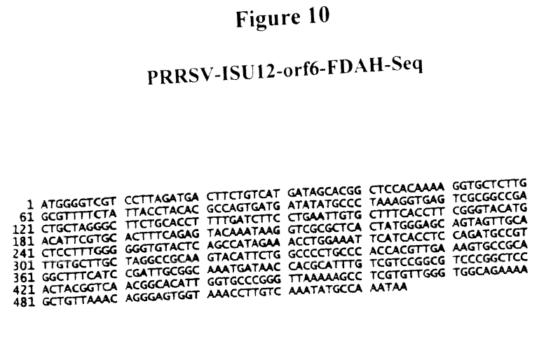

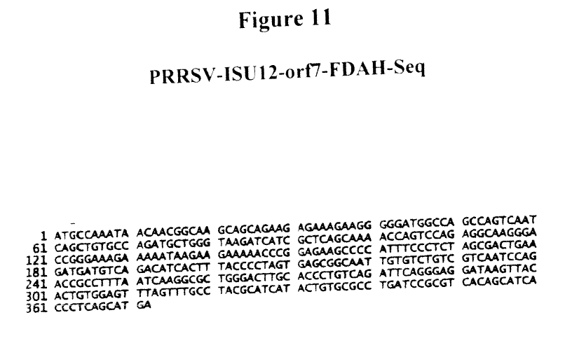

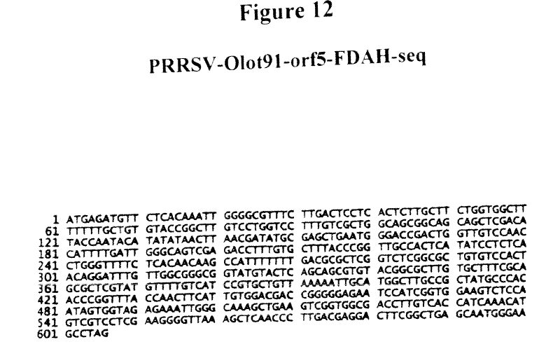

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”“an”and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Byrd, C M and Hruby, D E, Methods in Molecular Biology, Vol. 269: Vaccinia Virus and Poxvirology, Chapter 3, pages 31-40; Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated herein by reference in their entirety.

### DEFINITIONS

The terms used herein have the meanings recognized and known to those of skill in the art, however, for convenience and completeness, particular terms and their meanings are set forth below.

The term “about” means within 20%, more preferably within 10% and more preferably within 5%.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Depending on the circumstances, a primary challenge with an antigen alone, in the absence of an adjuvant, may fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

The term “antigen” refers to a compound, composition, or immunogenic substance that can stimulate the production of antibodies or a T-cell response in an animal, including compositions that are injected or absorbed into an animal. The term may be used to refer to an individual macromolecule or to a homogeneous or heterogeneous population of antigenic macromolecules. An antigen reacts with the products of specific humoral or cellular immunity. The term “antigen” broadly encompasses moieties including proteins, polypeptides, antigenic protein fragments, nucleic acids, oligosaccharides, polysaccharides, organic or inorganic chemicals or compositions, and the like. Furthermore, the antigen can be derived or obtained from any virus, bacterium, parasite, protozoan, or fungus, and can be a whole organism. The term “antigen” includes all related antigenic epitopes. Similarly, an oligonucleotide or polynucleotide, which expresses an antigen, such as in nucleic acid immunization applications, is also included in the definition. Synthetic antigens are also included, for example, polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. and Cell Biol. 75:402 408; Gardner et al. (1998) 12th World AIDS Conference, Geneva, Switzerland, Jun. 28 Jul. 3, 1998).

“Encoded by” or “encoding” refers to a nucleic acid sequence which codes for a polypeptide sequence, wherein the polypeptide sequence contains an amino acid sequence of at least 3 to 5 amino acids, more preferably at least 8 to 10 amino acids, and even more preferably at least 15 to 20 amino acids, a polypeptide encoded by the nucleic acid sequences. Also encompassed are polypeptide sequences, which are immunologically identifiable with a polypeptide encoded by the sequence. Thus, an antigen “polypeptide,” “protein,” or “amino acid” sequence may have at least 70% similarity, preferably at least about 80% similarity, more preferably about 90-95% similarity, and most preferably about 99% similarity, to a polypeptide or amino acid sequence of an antigen.

The term “exogenous” as used herein is intended to encompass that which is produced, originated, derived or developed outside the raccoon poxvirus genome, i.e., originating externally to the poxvirus genome such as, for example, the gene material obtained or isolated from the PRRSV that is considered foreign to the raccoon poxvirus genome.

A “gene” as used in the context of the present invention is a sequence of nucleotides in a nucleic acid molecule (chromosome, plasmid, etc.) with which a genetic function is associated. A gene is a hereditary unit, for example of an organism, comprising a polynucleotide sequence (e.g., a DNA sequence for mammals) that occupies a specific physical location (a “gene locus” or “genetic locus”) within the genome of an organism. A gene can encode an expressed product, such as a polypeptide or a polynucleotide (e.g., tRNA). Alternatively, a gene may define a genomic location for a particular event/function, such as the binding of proteins and/or nucleic acids (e.g., phage attachment sites), wherein the gene does not encode an expressed product. Typically, a gene includes coding sequences, such as polypeptide encoding sequences, and non-coding sequences, such as promoter sequences, poly-adenlyation sequences, transcriptional regulatory sequences (e.g., enhancer sequences). Many eucaryotic genes have “exons” (coding sequences) interrupted by “introns” (non-coding sequences). In certain cases, a gene may share sequences with another gene(s) (e.g., overlapping genes).

An “immune response” to an antigen or vaccine composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4⁺ T helper cells or CD8⁺ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376.

An “immunogenic ORF” or “immunogenic orf” refers to an open reading frame that elicits an immune response.

An “immunologically effective amount” as used herein refers to the amount of antigen or vaccine sufficient to elicit an immune response, either a cellular (T cell) or humoral (B cell or antibody) response, as measured by standard assays known to one skilled in the art. For example, with respect to the present invention, an “immunologically effective amount” is a minimal protection dose (titer) of ≧4.5 to 7.5 Log₁₀TCID₅₀/mL. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum. Furthermore, the level of protection of the immune response may be measured by challenging the immunized host with the antigen that has been injected. For example, if the antigen to which an immune response is desired is a virus or a tumor cell, the level of protection induced by the “immunologically effective amount” of the antigen is measured by detecting the percent survival or the percent mortality after virus or tumor cell challenge of the animals.

As defined herein “a non-essential site” in the raccoon poxvirus genome means a region in the viral genome, which is not necessary for viral infection or replication. Examples of non-essential sites in the raccoon poxvirus genome include, but are not limited to, the thymidine kinase (TK) site, the hemagglutinin (HA) site and the serine protease inhibitor site. The TK site of raccoon poxvirus is described in C. Lutze-Wallace, M. Sidhu and A. Kappeler, Virus Genes 10 (1995), pp. 81-84. The sequence of the TK gene of raccoon poxvirus can also be found in PubMed accession numbers DQ066544 and U08228. The HA site of raccoon poxvirus is described in Cavallaro K F and Esposito, J J, Virology (1992), 190(1): 434-9. The sequence of the HA gene of raccoon poxvirus can also be found in PubMed accession number AF375116.

The term “nucleic acid molecule” or “nucleic acid sequence” has its plain meaning to refer to long chains of repeating nucleotides such as the repeated units of purine and pyrimidine bases that direct the course of protein synthesis, that is, they encode and express the protein substance. As the term is used in the claims, the nucleic acid refers to the known exogenous or foreign genes that encode the porcine reproductive and respiratory syndrome virus proteins, desirably, the open reading frame genes.

The term “open reading frame” or “ORF”or “orf”, as used herein, refers to the minimal nucleotide sequence required to encode a particular virus protein without an intervening stop codon.

“Operably linked” refers to an arrangement of elements wherein the components so described are configured so as to perform their usual function. Thus, a given promoter that is operably linked to a coding sequence (e.g., a sequence encoding an antigen or interest) is capable of effecting the expression of the coding sequence when the regulatory proteins and proper enzymes are present. In some instances, certain control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence. Thus, a coding sequence is “operably linked” to a transcriptional and translational control sequence in a cell when RNA polymerase transcribes the coding sequence into mRNA, which is then trans-RNA spliced and translated into the protein encoded by the coding sequence.

The terms “porcine” and “swine” are used interchangeably and refer to any animal that is a member of the family Suidae such as, for example, a pig.

A “protective” immune response refers to the ability of a vaccine to elicit an immune response, either humoral or cell mediated, which serves to protect the mammal from an infection. The protection provided need not be absolute, i.e., the infection need not be totally prevented or eradicated, if there is a statistically significant improvement compared with a control population of porcine mammals, e.g. infected animals not administered the vaccine. Protection may be limited to mitigating the severity or rapidity of onset of symptoms of the infection.

The term “recombinant” as used herein simply refers to the raccoon poxvirus constructs that are produced by standard genetic engineering methods.

The term “replicable” refers to a microorganism, for example, a virus such as the raccoon poxvirus that is capable of replicating, duplicating or reproducing in a suitable host cell.

The terms “vaccine” or “vaccine composition” are used interchangeably herein and refer to a pharmaceutical composition comprising at least one immunologically active component that induces an immune response in an animal, and/or protects the animal from disease or possible death due to an infection, and may or may not include one or more additional components that enhance the immunological activity of the active component. A vaccine may additionally comprise further components typical to pharmaceutical compositions.

A “vector” is a DNA molecule, capable of replication in a host organism, into which a gene is inserted to construct a recombinant DNA molecule.

### GENERAL DESCRIPTION

In accordance with the present invention, there is provided a unique recombinant poxvirus vector in which the vector comprises one or more exogenous nucleic acid molecules encoding a porcine reproductive and respiratory syndrome virus protein. In one embodiment, the poxvirus vector may encode at least two copies of the same porcine reproductive and respiratory syndrome virus protein. The two copies of the same protein may be encoded by two different nucleic acid molecules or the two copies of the same protein may be encoded by the same nucleic acid molecule. The two copies of the same protein may be operably linked to one promoter or to separate promoters.

The proteins may be encoded by an open reading frame such as ORF1a or ORF1b to ORF7, preferably the ORF5, ORF6 and/or ORF3/ORF4/ORF7, of one or more porcine reproductive and respiratory syndrome virus (PRRSV) isolates with or without one or more open reading frames such as ORF1 to ORF13, preferably ORF2, of porcine circovirus type 2 (PCV-2) at the hemagglutinin (ha) and/or thymidine kinase (tk) loci, respectively. Another third non-essential site of the poxvirus genome for insertion of any of the nucleic acid molecules described herein is the serine protease inhibitor site. In one particular embodiment, the two or more exogenous nucleic acid molecules encode two or more open reading frames of porcine reproductive and respiratory syndrome virus selected from the group consisting of ORF5-ORF6, ORF5-ORF6-ORF7, ORF3-ORF5-ORF6, ORF3-ORF4-ORF7, ORF3-ORF4-ORF5-ORF7, ORF3-ORF4-ORF6-ORF7, and ORF3-ORF4-ORF5-ORF6-ORF7.

Also inserted at ha and/or tk loci is the gene expressing the glycoprotein of the feline leukemia virus (FeLV) that bears the FeLV P27⁺ phenotype (blue color in FeLV P27 ELISA). In these constructs, the FeLV P27⁺ or PCV-2 ORF2 (capsid gene) is used as a unique marker to screen the clones in different combinations. Preferred PRRSV isolates comprise the ISU12 (Iowa) and Olot/91 (Spanish) strains but other isolates may be readily substituted for the strains illustrating the invention in the examples below.

The object of the present invention, therefore, is to express four or more open reading frames (ORFs) of the U.S. and E.U. strains of the porcine reproductive and respiratory syndrome virus (PRRSV) alone or in combination with the ORF of porcine circovirus (PCV2) at the hemagglutinin (ha) and/or the thymidine kinase (tk) locus of the raccoon poxvirus (RCNV) genome in one viral construct to make a potent vaccine.

Another object of the invention is to use a new clone screening system that utilizes either limited dilution and blue-to-white plaque approach or limited dilution and virus ELISA as the screening marker rather than a conventional screening marker like LacZ.

The foregoing objects are accomplished by providing the novel vaccine comprising a recombinant raccoon poxvirus vector (rRCNV) in which the rRCNV expresses multiple open reading frames, preferably the ORF5, ORF6 and/or ORF3/ORF4/ORF7, of one or more PRRSV isolates with or without an open reading frame, preferably ORF2, of PCV-2 at the hemagglutinin (ha) and/or thymidine kinase (tk) loci, respectively.

Previous expression systems have been limited in their ability to express only one or two open reading frames (ORFs) of the PRRSV genes in one viral construct. The present invention advantageously permits the expression of four or more ORFs of the U.S. and E.U. strains of porcine reproductive and respiratory syndrome virus (PRRSV) alone or in combination with the open reading frame of porcine circovirus type 2 (PCV-2). Heretofore not described in the art, the superior expression system of this invention allows the insertion of multiple ORFs from PRRSV into the ha and/or tk loci and/or serine protease inhibitor locus of the raccoon poxvirus (RCNV) genome in one viral construct thereby creating a potent recombinant vaccine to protect pigs against PRRS.

The present invention uses a screening system that utilizes either limiting dilution and the blue-to-white plaque approach or limiting dilution and an ELISA as a screening marker. Quite beneficially, this screening system affords multiple site expression in one viral construct.

Raccoon poxvirus (Herman strain) was first isolated from the respiratory tract of raccoons with no clinical symptoms by Y. F. Herman in Aberdeen, Md. in 1961-1962 (Y. F. Herman, “Isolation and characterization of a naturally occurring pox virus of raccoons,” In: Bacteriol. Proc., 64th Annual Meeting of the American Society for Microbiology, p. 117 (1964)). In the rRCNV-PRRS construct of the present invention, the insertion of multiple open reading frames such as, for example, ORF5, ORF6 and/or ORF3/ORF4/ORF7, of one or more strains of PRRSV genes into the ha and/or tk loci of the raccoon poxvirus (RCNV) genome further attenuates the avirulent Herman strain. Advantageously, the rRCNV vectored PRRSV vaccine of the present invention also improves employee safety during vaccine production and completely eliminates any chance of the pathogenic virus surviving inactivation and decontamination procedures used during commercial production.

It is contemplated that the scientific advancement and novel method described in the present invention may be applied by the person having ordinary skill in the art to antigenic proteins of multiple reading frames obtained from PRRSV strains other than ISU12 and Olot/91 and can be inserted at the tk and/or ha loci of the raccoon poxvirus vector to provide an immunogenic vaccine for the protection against PRRS. For instance, other strains such as the North American strain VR-2332, the European strain Lelystad Virus and the like may readily substitute for the ISU12 and/or Olo 91 strains. Since the RNA virus is subject to mutations, alternatively, the desired ORF gene of a new field isolate or virus mutant may be isolated and utilized in the recombinant vaccine of the invention. Nevertheless, there is high homology between many mutants and numerous isolates of PRRSV found to date and, thus, using certain open reading frames such as the ORF5 or ORF6 in the vaccine construct of the invention has the advantage of being significantly effective against PRRS. For those isolates with lower homology around 40%, the practical advantage of the construct of the invention is that it can readily accommodate the antigenic proteins of several ORFs from multiple viral sources to provide an efficacious, broad-spectrum vaccine. Such prevention of PRRS through a successful inoculation program of piglets is of extreme importance to the pig industry as there is no effective long term treatment for the disease.

It is further contemplated that the poxvirus vector useful in the invention may comprise, but is not limited to, the raccoon poxvirus, the canary poxvirus, the fowlpox virus, the swinepox virus, the vaccinia virus and the like but desirably the raccoon poxvirus is employed. While an inactivated poxvirus vector is useable in the practice of the method of the present invention, a live vector is preferred.

This invention also provides a new method of protecting pigs against Postweaning Multisystemic Wasting Syndrome (PMWS) and/or Porcine Reproductive and Respiratory Syndrome (PRRS) by administering the potent new recombinant vaccine to pigs in need of protection.

In the method of the invention, an immunologically effective amount of the vaccines of the present invention is administered to a pig, particularly young piglets, in need of protection against PMWS and/or PRRS in order to induce a protective immune response in the animals. An effective immunizing amount given to the pig is one in which a sufficient immunological response to the vaccine is attained to protect the pig from the harmful effects of PCV-2 and/or PRRSV. A protective immune response is considered to be obtained when the vaccine is able to protect at least a significant number of the inoculated pigs as required by standard values in the vaccine field. The immunologically effective dosage or the effective immunizing amount that inoculates the animal and elicits satisfactory vaccination effects can be easily determined or readily titrated by routine testing such as, for example, by standard titration studies.

The novel vaccine constructs of the present invention are employed for the vaccination of healthy piglets at approximately three months of age for the prevention of Postweaning Multisystemic Wasting Syndrome (PMWS) and/or Porcine Reproductive and Respiratory Syndrome (PRRS). The vaccine may also be given to mature or adult sows (i.e., older than three months) prior to breeding. The vaccine can be administered in a single dose or in repeated doses if antibody titers decline and a booster shot is deemed necessary. Desirably, the vaccine is administered to healthy animals in a single inoculation to provide long term protection against disease, protecting the animals for at least one year to three years or longer. Appropriate dosages are determined by standard dose titration studies. The vaccine may contain an immunologically effective amount of any one of the recombinant raccoon poxvirus vectored constructs described herein. In another embodiment, the vaccine may contain an immunologically effective amount of any two or more of the recombinant raccoon poxvirus vector constructs described herein.

The vaccine can be readily given by any route of administration, orally, intranasally, transdermally (i.e., applied on or at the skin surface for systemic absorption), parenterally, etc though some routes may be logistically more difficult depending on the particular animal and the handler. The parenteral route of administration includes, but is not limited to, subcutaneous, intramuscular, intravenous, intraperitoneal, intradermal (i.e., injected or otherwise placed under the skin) routes and the like. Preferably, the vaccine is administered subcutaneously.

The poxvirus vector may be live or inactivated by conventional procedures for preparing inactivated viral vaccines, for example, using BEI (binary ethyleneimine), formalin and the like, with BEI being a preferred inactivant, though it is highly desirable for the vaccine of the present invention to use a live raccoon poxvirus for optimal and potent immunological efficacy.

Beneficially, the live recombinant RCNV expressing multiple ORFs of PCV-2 and/or PRRSV are useful as a vaccine, either alone or in combination with suitable carriers, diluents and adjuvants. The vaccine product may optionally contain a variety of typical, non-toxic, pharmaceutically acceptable additives, diluents and adjuvants that include, but are not limited to, preservatives; stabilizers; emulsifiers; aluminum hydroxide; aluminum phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminum phosphate, dextran sulfate, DEAE-Dextran and the like; carboxypolymethylene, such as CARBOPOL® (polyacrylic polymer commercially available from B.F. Goodrich Company, Cleveland, Ohio); ethylene maleic anhydride or ethylene/maleic anhydride copolymers (EMA®, a linear ethylene/maleic anhydride copolymer having approximately equal amounts of ethylene and maleic anhydride, having an estimated average molecular weight of about 75,000 to 100,000, commercially available from Monsanto Co., St. Louis, Mo.); acrylic copolymer emulsions such as a copolymer of styrene with a mixture of acrylic acid and methacrylic acid like NEOCRYL® A640 (e.g. U.S. Pat. No. 5,047,238, an uncoalesced aqueous acrylic acid copolymer of acrylic acid and methacrylic acid mixed with styrene, commercially available from Polyvinyl Chemicals, Inc., Wilmington, Mass.); bacterial cell walls such as mycobacterial cell wall extract; Corynebacterium-derived adjuvants such as Corynebacterium parvum; Propionibacterium-derived adjuvants such as Propionibacterium acne; Mycobacterium bovis (Bacille Calmette-Guerin, or BCG); subviral particle adjuvants such as orbivirus; cholera toxin; N,N-dioctadecyl-N′,N′-bis(2-hydroxyethyl)-propanediamine (avridine); monophosphoryl lipid A; dimethyidioctadecylammonium bromide (DDA, commercially available from Kodak, Rochester, N.Y.); synthetics and mixtures thereof. Further additives that may be employed in the present vaccine include, but are not limited to, dextrose, conventional antioxidants and conventional chelating agents, such as ethylenediamine tetraacetic acid (EDTA). Other pharmaceutically acceptable adjuvants that may optionally supplement the vaccine formulation include, but are not limited to, polyanions, polycations, peptides, mineral oil emulsion, immunomodulators, a variety of combinations and the like. Further non-limiting examples of suitable adjuvants include squalane and squalene (or other oils of animal origin); polyoxyethylene-polyoxypropylene block copolymers such as PLURONIC® (L121, for example, commercially available from BASF Aktiengesellschaft, Ludwigshafen, Germany); saponin; Quil A (commercial name of a purified form of Quillaja saponaria, available from Iscotec AB, Sweden and Superfos Biosector a/s, Vedbaek, Denmark); mineral oils such as MARCOL® (a purified mixture of liquid saturated hydrocarbons, commercially available from Exxon-Mobil, Fairfax, Va.); vegetable oils such as peanut oil; interleukins such as interleukin-2 and interleukin-12; interferons such as gamma interferon; animal poxvirus proteins or mixtures thereof. Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, nontoxic oils useful for injectables or intranasal vaccines. Desirably, aluminum hydroxide is admixed with other adjuvants such as saponin or Quil A to form combinations such as saponin-aluminum hydroxide or Quil A-aluminum hydroxide.

A preferred adjuvant comprises ethylene/maleic anhydride copolymer, copolymer of styrene with a mixture of acrylic acid and methacrylic acid, mineral oil emulsion or combinations thereof. A particularly preferred adjuvant system that significantly enhances the potency of the rRCNV-PRRS construct of the present invention comprises the combination of an ethylene/maleic copolymer such as EMA-31® (a linear ethylene/maleic anhydride copolymer having approximately equal amounts of ethylene and maleic anhydride, having an estimated average molecular weight of about 75,000 to 100,000, commercially available from Monsanto Co., St. Louis, Mo.) and an acrylic acid copolymer emulsion such as NEOCRYL® (an uncoalesced aqueous acrylic acid copolymer of acrylic acid and methacrylic acid mixed with styrene (acrylic-styrene emulsion), commercially available from Polyvinyl Chemicals, Inc., Wilmington, Mass.).

The recombinant vaccine composition of the present invention may also optionally contain a mixture with one or more additional porcine antigens such as, for example, Mycoplasma hyopneumoniae, Haemophilus parasuis, Pasteurella multocida, Streptococcum suis, Actinobacillus pleuropneumoniae, Bordetella bronchiseptica, Salmonella choleraesuis, Erysipelothrix rhusiopathiae, leptospira bacteria, swine influenza virus (SIV), porcine parvovirus, Escherichia coli, porcine respiratory coronavirus, rotavirus, a pathogen causative of Aujezky's Disease, Swine Transmissible Gastroenteritis, etc.

Any method known to those skilled in the art may be used to prepare the genetic constructs of the present invention. For example, advantage may be taken of particular restriction sites for insertion of any of the desired nucleic acid sequences into the raccoon poxvirus vector using standard methodologies. Alternatively, one may utilize homologous recombination techniques when the insertion of large sequences is desired, or when it is desirable to insert multiple genes, as described herein. In this method, the plasmid sequences flanking the insertion site into which are to be inserted multiple genes, contain sequences which have sufficient homology with sequences present in the raccoon poxvirus genome to mediate recombination. The flanking sequences must be homologous to a region of the raccoon poxvirus that is non-essential for the growth and propagation of the raccoon poxvirus, such as the hemagglutinin locus, or the thymidine kinase locus, or the serine protease inhibitor locus. Although one promoter may be used to drive the expression of two exogenous genes to be recombined, the use of two promoters in an insertion vector, each promoter operably linked to an individual gene will also provide efficient expression.

Generally, the virus rRCNV-PRRS may be constructed by a blue-to-white plaque approach, for instance, by following five key steps: (1) Clone ORF5, ORF6, ORF7 or ORF3 of PRRSV ISU12 (Iowa) and Olot/91 (Spanish isolate) strains into plasmids pFD2000A and/or pFD2003SEL; (2) Construct the plasmid pFD2000A PRRS ORF7 (P₁₁)-ORF5 (Olot) (P_{SEL})-ORF6 (P_{SEL})-ORF5 (P_{SEL}) or the plasmid pFD2000A PRRS ORF3 (P₁₁)-ORF5 (Olot) (P_{SEL})-ORF6 (P_{SEL})-ORF5 (P_{SEL}); (3) Create pool clones by three-way infection/transfection: COS7 cells, plasmid at Step 2 and rRCNV-FeLV; (4) Clone screening by limited dilution and blue-to-white plaque approach; and (5) Determine molecular characterization of rRCNV-PRRS_{btw} by PCR, ELISA and Western blot. Alternatively, a white-to-blue plaque approach may be used in which the following five key steps are performed: (1) Clone ORF5 and ORF6 of PRRSV ISU12 and Olot/91 strains, FeLV P27 into plasmids pFD2000A and/or pFD2003SEL; (2) Construct the plasmid pFD2000A FeLV P27 (P₁₁)-PRRS ORF5 (Olot) (P_{SEL})-ORF6 (P_{SEL})-ORF5 (P_{SEL}) or pFD2000A PRRS ORF5 (Olot) (P_{SEL})-ORF6 (P_{SEL})-ORF5//LacZ (P_{7.5}); (3) Create pooled clones by three-way infection/transfection: COS7 cells, plasmid at Step 2 and RCNV; (4) Clone screening by limited dilution and FeLV P27 ELISA or LacZ; and (5) Determine molecular characterization of rRCNV-PRRS_{wtb} by PCR, ELISA and Western blot.

The rRCNV-PRRS-PCV of the present invention may be constructed by way of the following five key steps: (1) Clone ORF5, ORF6, ORF3 of PRRSV ISU12 and Olot/91 strains and PCV2 ORF2 (capsid gene) into plasmids pFD2000A and/or pFD2003SEL; (2) Construct the plasmid pFD2000A PRRS ORF3 (P₁₁)-ORF5 (Olot) (P_{SEL})-ORF6 (P_{SEL})-ORF5 (P_{SEL})-PCV2 ORF2 (P₁₁ or P_{SEL}); (3) Create pooled clones by three-way infection/transfection: COS7 cells, plasmid at Step 2 and RCNV; (4) Clone screening by limited dilution and PCV2 ELISA (as screening marker); and (5) Determine molecular characterization of rRCNV-PRRS_{pcv} by PCR, ELISA and Western blot. Alternatively, a white-to-blue plaque approach may be used in which the following four key steps are performed: (1) Clone PCV2 ORF2 into the plasmid pFD2001TK or pFD2006TK to generate the plasmid pFD2001TK PCV ORF2; (2) Create pooled clones by three-way infection/transfection: COS7 cells, plasmid at Step 1 and rRCNV-PRRS ORF3 (P₁₁)-ORF5 (Olot) (P_{SEL})-ORF6 (P_{SEL})-ORF5 (P_{SEL}); (3) Clone screening by limited dilution and PCV2 ELISA or LacZ; and (5) Determine molecular characterization of rRCNV-PRRS//PCV by PCR, ELISA and Western blot.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

### Example 1

Construction of rRCNV-PRRS (Blue-to-White Plaque Approach)

The rRCNV-PRRS_{btw} was constructed by the following five key steps: (1) Clone ORF5, ORF6, ORF7 or ORF3 of PRRSV ISU12 (Iowa) and Olot/91 (Spanish isolate) strains into plasmids pFD2000A and/or pFD2003SEL; (2) Construct the plasmid pFD2000A PRRS ORF7 or ORF3 (P₁₁)-ORF5 (Olot) (P_{SEL})-ORF6 (P_{SEL})-ORF5 (P_{SEL}); (3) Create pool clones by three-way infection/transfection: COS7 cells, plasmid at Step 2 and rRCNV-FeLV; (4) Clone screening by limited dilution and blue-to-white plaque approach; and (5) Determine molecular characterization of rRCNV-PRRS_{btw} by PCR, ELISA and Western blot.

The blue-to-white plaque screening approach uses the parent virus rRCNV-FeLV bearing the FeLV P27⁺ phenotype (blue color in FeLV P27 ELISA). Through homologous recombination of RCNV HA flanking sequence between rRCNV-FeLV and pFD200A PRRS ORF7-ORF5 (Olot)-ORF6-ORF5, the phenotype of recombinant rRCNV-PRRS will change from blue to white plaque because PRRSV ORFs are inserted at ha locus to replace the sequence of FeLV gag-env gp85 in the parent strain (rRCNV-FeLV-blue plaque).

### Example 2

Construction of rRCNV-PRRS (Blue-to-White Plaque Approach)

The rRCNV-PRRS_{wtb} was constructed by the following five key steps: (1) Clone ORF5 and ORF6 of PRRSV ISUI2 and Olot/91 strains, FeLV P27 into plasmids pFD2000A and/or pFD2003SEL; (2) Construct the plasmid pFD2000A FeLV P27 (P₁₁)-PRRS O RF5 (Olot) (P_{SEL})-ORF6 (P_{SEL})-ORF5 (P_{SEL}); (3) Create pooled clones by three-way infection/transfection: COS7 cells, plasmid at Step 2 and RCNV; (4) Clone screening by limited dilution and FeLV P27 ELISA or LacZ; and (5) Determine molecular characterization of rRCNV-PRRS_{wtb} by PCR, ELISA and Western blot.

The white-to-blue plaque screening approach uses the recombinant virus rRCNV-PRRS_{wtb} bearing the FeLV P27⁺or LacZ phenotype (blue color in FeLV P27 ELISA or LacZ⁺ phenotype) while wild type RCNV shows white color in FeLV P27 ELISA or LacZ staining.

### Example 3

Construction of rRCNV-PRRS-PCV

The rRCNV-PRRS_{pcv} is constructed by the following five key steps: (1) Clone ORF5, ORF6, ORF3 of PRRSV ISU12 and Olot/91 strains and PCV2 ORF2 (capsid gene) into plasmids pFD2000A and/or pFD2003SEL; (2) Construct the plasmid pFD2000A PRRS ORF3 (P₁₁)-ORF5 (Olot) (P_{SEL})-ORF6 (P_{SEL})-ORF5 (P_{SEL})-PCV² ORF2(P₁₁ or P_{SEL}); (3) Create pool clones by three-way infection/transfection: COS7 cells, plasmid at Step 2 and RCNV; (4) Clone screening by limited dilution and PCV2 ELISA (as screening marker); and (5) Determine molecular characterization of rRCNV-PRRS_{pcv} by PCR, ELISA and Western blot.

The PCV2 approach uses the PCV-2 capsid ELISA as a unique marker for screening of clones, and to create a new combination vaccine of rRCNV-PRRS/PCV in one viral construct.

Alternatively, the rRCNV-PRRS_{pcv} is constructed by the following four key steps: (1) Clone PCV2 ORF2 (capsid gene) into plasmids pFD2001TK and/or pFD2006TK to generate the plasmid pFD2001TK PCV2 ORF2 (P₁₁ or P_{SEL}); (2) Create pool clones by three-way infection/transfection: COS7 cells, plasmid at Step 1 and rRCNV-PRRS; (3) Clone screening by limited dilution and PCV2 ELISA (as screening marker); and (5) Determine molecular characterization of rRCNV-PRRS//PCV by PCR, ELISA and Western blot.

### Example 4

Efficacy Study of rRCNV-PRRS Vaccine Candidate in Pigs

The above constructs (rRCNV-PRRS_{btw}, rRCNV-PRRS_{wtb} and rRCNV-PRRS_{pcv}) are formulated with or without an immune enhancing adjuvant and evaluated for vaccine efficacy in pigs. The successful results of the challenge studies will show the efficacy of the recombinant PRRS, PCV or combination vaccine in pigs.

### Example 5

Construction of rRCNV-PRRSV

Briefly, the virus rRCNV-PRRSV was constructed by insertion of envelope glycoprotein (orf 5) gene and matrix protein (orf 6) gene of the porcine respiratory and reproductive syndrome virus (PRRSV) ISU 12 (US strain) and the envelope glycoprotein (orf 5) gene of PRRSV Olot 91 (EU strain) genes into the hemagglutination (ha) locus of the RCNV genome, an avirulent Herman strain.

The construction processes of rRCNV-PRRSV were provided through two major steps. First, the PCR-amplified 603-bp orf 5 and 525-bp orf6 of PRRSV ISU 12 strain and 606-bp orf 5 of PRRSV Olot 91 strain were subcloned into a plasmid pFD2003SEL vector (containing a lacZ reporter gene under the control of promoter P11) to generate plasmid pFD2003SEL-PRRSV Olot 91 orf5-(SEL)-ISU 12 orf 6 (SEL)-ISU 12 orf 5 (SEL). Both PRRSV ISU 12 orf 5 and orf 6 and PRRSV Olo 91 orf 5 genes are co-expressed under the control of promoter P_{SEL}. Second, three-way co-infection/transfection of rRCNV-FCV (2280-DD1) and plasmid pFD2003SEL-PRRSV Olot 91 orf5-(SEL)-ISU 12 orf 6 (SEL)-ISU 12 orf 5 (SEL) in COS-7 cells was conducted to generate rRCNV-PRRSV by allelic exchange at the ha locus. The blue plaques (Lac⁺) were cloned by five successive rounds of plaque purification in Vero cells. The clone candidates were further expanded two more times in Vero cells using Minimum Essential Medium (MEM) supplemented with 0.05% lactalbumin hydrolysate (LAH), 30 μg/mL gentamicin sulfate and 5% fetal bovine serum, and thereafter confirmed by gene-specific PCR. The seventh passage was used to prepare a pre-master seed. The Master Seed was established by a 1:20,000 dilution of pre-master seed, and designated rRCNV-PRRSV, in which the raccoon poxvirus as a live vector is capable of expressing the PRRSV envelope glycoproteins (orf5) and matrix protein (M) of PRRSV ISU 12 and Olot 91 strains at the ha locus, respectively. The master seed virus of the PRRSV vaccine, live raccoon poxvirus vector (rRCNV-PRRSV) was identified by PRRSV orf5 or orf6 gene(s)-specific PCR testing. Immunogenicity studies of porcine respiratory and reproductive syndrome virus vaccine, live/inactivated raccoon poxvirus vector alone or in combination with Suvaxyn PCV2 One Dose and or Suvaxyn MH-one, or live chimeric cPCV1-2 and or modified live Mycoplasma hyopenumoniae (for example, J strain) are being conducted in piglets and sows.

### Example 6

Immunogenicity Study of Porcine Reproductive and Respiratory Syndrome Virus Vaccine, Live Raccoon Poxvirus Vector Alone or in Combination with Suvaxyn PCV2 One dose, in Piglets

To demonstrate the efficacy of the PRRSV fraction, pigs will be administered the rRCNV-PRRSV vaccine (lyophilized cake) and adjuvant-containing sterile water or Suvaxyn PCV2 One Dose as diluent in two-dose regimen at a 2-3 week interval, followed by virulent PRRSV challenge or PRRSV/PCV2 co-challenge 2-3 weeks post the second vaccination. The efficacy of the rRCNV-PRRSV fraction will be evaluated based on the reduction of PRRSV viremia and reduction of lung lesions caused by PRRSV. Suvaxyn® PCV2 One contains inactivated Porcine Circovirus Type 1-Type 2 Chimera (cPCV1-2) formulated with FDAH's proprietary adjuvant.

### Materials and Methods

### Test Animals

No fewer than 40 (20 vaccinates and 20 controls) mixed breed male and female conventional pigs, 3 weeks of age or older, will be used for this study. The pigs for the study will be purchased from a commercial farm and identified using an ear tag. Pigs will be obtained from a single source herd. At the time of the first vaccination pigs will be seronegative to PRRSV as determined by IDEXX HerdCheck ELISA kits (S/P ratio <0.4).

### Test Vaccines

### Composition of Test Vaccines

Lyophilized fraction: The vaccine consists of live rRCNV-PRRSV. The vaccine's virus titer will be titrated in five replicate assays in order to establish the dose administrated in the study. Liquid fraction: The diluent will be Suvaxyn PCV2 One Dose or 20% SL-CD adjuvant. The lyophilized fraction of rRCNV-PRRSV will be rehydrated with the liquid fraction at the time of vaccination.

### Study Design

The proposed studies will be randomized complete block designs (superiority trial). Litters will be allocated to each study replicate. The piglets within litters in each study will then be divided into two groups as follows:

| | | Number of |
|---|---|---|
| Group | Vaccine | Animals |
| VI vaccinates | Lyophilized rRCNV-PRRSV | ≧20 |
| | (cake) + Suvaxyn PCV2 | |
| | One Dose | |
| C1-Controls | Suvaxyn PCV2 One Dose | ≧20 |
| | (Diluent) | |
| V2-Vaccinates | Lyophilized rRCNV-PRRSV | ≧20 |
| | (Cake) + Water-SL-CD | |
| | Adjuvant (Diluent) | |
| C2-Controls | Water-SL-CD Adjuvant | ≧20 |
| | (Diluent) | |

### Vaccination

The vaccination route will be intramuscular on the right side of neck.

For pigs in Study 1: group V1 will be given two 2.0 mL doses of the rRCNV-PRRSV+Suvaxyn PCV2 One Dose; pigs in control group C1 will be given two 2.0 mL doses of Suvaxyn PCV2 One Dose.

For pigs in Study 2: group V2 will be given two 2.0 mL doses of rRCNV-PRRSV+Water/SL-CD; pigs in control group C2 will be given two 2.0 mL doses of Water-SL-CD Adjuvant.

The first vaccination will be administered at 3 weeks of age, and repeat vaccination will be 2-3 weeks later.

### Challenge

All animals will be virulently challenged two to three weeks following the second vaccination.

Pigs in groups V1 and C1 will be challenged with at least 2×10^{4.8} FAID₅₀ of the wild type pathogenic PCV2 (strain 40895) and with at least 2×10^{4.0} TCID₅₀ of PRRSV (ISU-12 or equivalent). These two challenge materials will be mixed together for at least 30 minutes prior to challenge. Challenge dose will be 4 mL intranasally (2 mL per nostril).

Pigs in groups V2 and C2 will be challenged with at least 2×10^{4.0} TCID₅₀ of PRRSV (ISU-12 or equivalent). Challenge dose will be 2 mL intranasally (1 mL per nostril). See Attachment 2 for challenge record.

The challenge viruses will be titrated by PCV2-specific and PRRSV-specific indirect immunofluorescence assays (IFA) on the day of challenge.

### Observation

All pigs will be observed for clinical signs and temperatures daily from −2 to 10 days post challenge (DPC). Any abnormal observation will be reported to the study director or plant veterinarian.

Monitored clinical signs may include, but not be limited to, inappetence, lethargy, depression, diarrhea, sneezing, coughing, nasal discharge, ocular discharge, dyspnea and death. If any pig dies during the post-challenge observation period, necropsy will be performed and the necropsy report will be attached to the final report.

### Sample Collection and Testing

### Sample Collection

### Nasal Swabs

Nasal swabs will be collected on 0 day post vaccination one (DPV1) for PRRSV and PCV2 isolation to ensure there is no PRRSV or PCV2 infection in the tested animals prior to vaccination.

The nasal swab samples will be placed into individual sterile tubes containing 3 mL of MEM with lactalbumin hydrolysate (LAH) and gentamicin (60 μg/mL), penicillin (100 U/mL) and streptomycin (100 μg/mL), and stored at or below −50° C. until tested.

### Serum Samples

Pigs will be bled for serum samples (no more than ten mL) at 0 DPV1, 0 DPV2, 7 DPV2, 14 DPV2,-1, 6, and 9 for ELISA testing; and at −1, 3, 6, and 9 for PCR testing.

### Necropsy

All animals will be necropsied at 10-11 DPC. The lungs will be collected for lung lesion scoring.

### Sample Testing

### Enzyme-Linked Immunosorbent Assay (ELISA)

Serum antibodies to PRRSV will be detected by IDEXX® HerdCheck ELISA kit (Westbrook, Me.). Briefly, serum samples are diluted 1:40 in Sample Diluent and 100 μL of each sample are added in duplicate (one well for PRRSV antigen and one well for normal host cell (NHC) antigen) to antigen-coated 96-well plates. After 30-minute incubation period at room temperature, liquid is decanted and the plates washed 3-5 times with approximately 300 μL of the phosphate-buffered wash solution. 100 μL of anti-Porcine-HRPO Conjugate is then added to all wells. Plates are incubated for 30-minutes at room temperature. After incubation period, plates are washed again as above. 100 μL of TMB Substrate Solution is then added to each well. Plates are incubated at room temperature for 15 minutes before 100 μL of Stop Solution is added to all wells to stop the reaction. The optical density (OD) is then measured at 650 nm on a microplate reader. The arbitrary serum titer will be reported as the sample/positive (S/P) ratio and is determined by taking the quantitiy of the sample OD-PRRSV minus the sample OD-NHC divided by the quantity of mean positive control-PRRSV minus the mean positive control-NHC.

### PCR Testing

PRRSV-specific RT-PCR testing will be used to detect the presence of PRRSV in serum. RNA-extraction will be performed on serum samples using QlAamp® Viral RNA Mini Kit (Valencia, Calif.).

### Lung Lesion Scoring

The lungs of each individual pig will be examined by two independent scorers for gross macroscopic lesions based on the amount of lung parenchyma affected by lesions. The scorers of the lesions will be blinded to the identity of the treatment for each pig. Briefly, the score for lung lesions in each lobe will be determined by estimating the percentage of the lobe exhibiting PRRSV-like lesions (based on color and texture) and multiplied by the number of points possible for that lobe. Maximum score for each lobe is determined by the relative percentage of the total lung volume occupied by the lobe. The scores from the dorsal and ventral aspects of all lobes will then be added together to obtain the total score for each pig. The maximum total score possible for each animal is 100.

### Primary Outcome

For the claim of reduction of lung lesions, total scores of lesions present on lungs will be assessed.

### Secondary Outcome

Secondary outcome will be the occurrence of PRRSV viremia.

### Statistical Analyses

### Estimator

For the evaluation of reduction in severity of lung lesions, the estimator will be the mitigated fraction (MF) statistic. The mitigated fraction will be calculated as:

$$M F = \frac{2 {W}_{1} - {n}_{c} ( 1 + {n}_{c} + {n}_{v} )}{{n}_{c} {n}_{v}}$$

where:
W₁=Wilcoxon Rank sum statistic
n_{c}=number of subjects in the control group
nᵥ=number of subjects in each vaccinated group
   *Note: the MF statistic will be estimated for each study replicate.

### Statement of Method of Calculating Interval Estimates

The 95% confidence interval for the mitigated fraction for each study replicate will be calculated.

### Hypothesis Statement

For the claim of reduction severity of lung lesions:

H_{O}:Mᵥ=M_{c}

H_{A}:Mᵥ≠M_{c}

where:
Mᵥ=Median of the lung lesion score rank in the vaccinated group
M_{c}=Median of the lung lesion score rank in the control group

These studies will test the null hypothesis that there is no difference in the severity (rank) in lung lesions between the vaccinated group and the placebo control group for each study replicate.

### Other Statistical Considerations

The distribution of pigs litter will be randomly assigned within litter.

### Statistical Analyses

### Statistical Methods

### Primary Outcome

The test repeatability of the score within animal will be assessed by intra class correlation. If the test repeatability is deemed sufficient, the mean lung score for each pig will be calculated by averaging the lung scores from each scorer. The calculated lung scores will be compared between the two treatment groups for each study replicate by Wilcoxon Rank Sum with the calculated lung score as the dependent variable and treatment included as an independent variable. The analysis will be stratified by litter if there are sufficient complete blocks. The HL shift will be estimated. The MF will be estimated from the Wilcoxon statistic.

### Secondary Outcome

A secondary outcome will be assessed for occurrence of viremia. A single occurrence of viremia will categorize pig as positive for viremia (Yes or No). Viremia will be compared between treatment groups in a generalized linear mixed model with the occurrence of viremia (Yes or No) as the binomial dependent variable and treatment included as an independent variable. If there are sufficient complete blocks, the litter will be included as a random effect covariate in the model.

All statistical analysis will be performed using the SAS system (SAS Institute, Inc.). The level of significance will be set at p<0.05

In the foregoing, there has been provided a detailed description of particular embodiments of the present invention for purpose of illustration and not limitation. It is to be understood that all other modifications, ramifications and equivalents obvious to those having skill in the art based on this disclosure are intended to be included within the scope of the invention as claimed.

What is claimed is:

1. A recombinant raccoon poxvirus vector (rRCNV) comprising one or more exogenous nucleic acid molecules encoding a porcine reproductive and respiratory syndrome virus (PRRSV) protein, wherein:
(a) at least one nucleic acid molecule is inserted into the hemagglutinin locus or the thymidine kinase locus of the raccoon poxvirus genome; or
(b) at least two nucleic acid molecules are inserted into the hemagglutinin locus or the thymidine kinase locus of the raccoon poxvirus genome; or
(c) at least one nucleic acid molecule is inserted into the hemagglutinin locus and at least one nucleic acid molecule is inserted into the thymidine kinase locus of the raccoon poxvirus genome.

2. The recombinant raccoon poxvirus vector of claim 1, wherein at least two nucleic acid molecules are inserted into the hemagglutinin locus and at least two nucleic acid molecules are inserted into the thymidine kinase locus of the raccoon poxvirus genome.

3. The recombinant raccoon poxvirus vector according to claim 1, further comprising a nucleic acid molecule encoding a porcine reproductive and respiratory syndrome virus (PRRSV) protein that is inserted into a third non-essential site of the raccoon poxvirus genome in addition to the thymidine kinase and the hemagglutinin loci of the raccoon poxvirus genome.

4. The recombinant raccoon poxvirus vector of claim 3, wherein the third non-essential site of the raccoon poxvirus genome is the serine protease inhibitor site.

5. The recombinant raccoon poxvirus vector of claim 1, wherein the poxvirus vector encodes at least two copies of the same porcine reproductive and respiratory syndrome virus protein.

6. The recombinant raccoon poxvirus vector of claim 5, wherein said two copies of the same protein are encoded by two different nucleic acid molecules.

7. The recombinant raccoon poxvirus vector of claim 5, wherein said two copies of the same protein are encoded by the same nucleic acid molecule.

8. The recombinant raccoon poxvirus vector of claim 7, wherein the nucleic acid molecule(s) encoding said two copies of the same protein are operably linked to one promoter.

9. The recombinant raccoon poxvirus vector of either one of claims 6 or 7, wherein the nucleic acid molecule(s) encoding said two copies of the same protein are operably linked to separate promoters.

10. The recombinant raccoon poxvirus vector of either of claims 6, or 7, wherein said two copies of the same protein are encoded by a porcine reproductive and respiratory syndrome virus (PRRSV) open reading frame (ORF) selected from the group consisting of ORF1, ORF2, ORF3, ORF4, ORF5, ORF6, ORF7 and a combination thereof.

11. The recombinant raccoon poxvirus vector of claim 1, wherein each of the exogenous nucleic acid molecules encodes a PRRSV ORF.

12. The recombinant raccoon poxvirus vector of claim 11, wherein the ORF is one or more selected from the group consisting of ORF1, ORF2, ORF3, ORF4, ORF5, ORF6, ORF7 and a combination thereof.

13. The recombinant raccoon poxvirus vector of claim 2, wherein said two or more exogenous nucleic acid molecules encode two or more PRRSV ORFs selected from the group consisting of ORF5-ORF6, ORF5-ORF6-ORF7, ORF3-ORF5-ORF6, ORF3-ORF4-ORF7, ORF3-ORF4-ORF5-ORF7, ORF3-ORF4-ORF6-ORF7, and ORF3-ORF4-ORF5-ORF6-ORF7.

14. The recombinant raccoon poxvirus vector according to claim 1, wherein the porcine reproductive and respiratory syndrome virus is ISU12 (Iowa), Olot/91 (Spanish) or both.

15. The recombinant raccoon poxvirus vector according to claim 1, wherein two or more nucleic molecules are inserted into the hemagglutinin locus of the raccoon poxvirus genome.

16. The recombinant raccoon poxvirus vector according to claim 15, further comprising a nucleic acid molecule encoding an open reading frame of porcine circovirus type 2 (PCV2).

17. The recombinant raccoon poxvirus vector according to claim 16, wherein the PCV2 open reading frame is ORF2.

18. The recombinant raccoon poxvirus vector according to claim 17, wherein the PCV2 ORF2 is separately inserted into the thymidine kinase locus of the raccoon poxvirus genome.

19. The recombinant raccoon poxvirus vector according to claim 15, further comprising a nucleotide sequence encoding a glycoprotein of feline leukemia virus P27⁺ phenotype.

20. The recombinant raccoon poxvirus vector according to claim 1, wherein the raccoon poxvirus is live and replicable.

21. A recombinant porcine vaccine comprising an immunologically effective amount of the recombinant raccoon poxvirus vector of claim 1 and, optionally, a suitable carrier, diluent or adjuvant.

22. A recombinant porcine vaccine comprising an immunologically effective amount of two or more of the recombinant raccoon poxvirus vectors of claim 1 and, optionally, a suitable carrier or diluent.

23. The recombinant porcine vaccine according to claim 21, wherein the raccoon poxvirus is live and replicable.

24. The recombinant porcine vaccine according to claim 21, further comprising at least one additional porcine antigen.

25. The recombinant porcine vaccine according to claim 24, wherein the additional porcine antigen is selected from the group consisting of Mycoplasma hyopneumoniae antigen, Haemophilus parasuis antigen, Pasteurella multocida antigen, Streptococcum suis antigen, Actinobacillus pleuropneumoniae antigen, Bordetella bronchiseptica antigen, Salmonella choleraesuis antigen, Erysipelothrix rhusiopathiae antigen, leptospira bacteria antigen, swine influenza virus antigen, porcine parvovirus antigen, Escherichia coli antigen, porcine respiratory coronavirus antigen, rotavirus antigen, an antigen from a pathogen causative of Aujesky's Disease, Swine Transmissible Gastroenteritis antigen and a combination thereof.

26. A method for inducing an immune response against porcine reproductive and respiratory syndrome virus, comprising administering to a porcine animal an immunologically effective amount of the recombinant porcine vaccine of claim 25.

27. The method according to claim 24, wherein the recombinant porcine vaccine comprises a live and replicable recombinant raccoon poxvirus vector.

28. A method for preventing porcine reproductive and respiratory syndrome and Postweaning Multisystemic Wasting Syndrome, comprising administering to a porcine animal in need of protection a recombinant porcine vaccine comprising an immunologically effective amount of the recombinant raccoon poxvirus vector of any one of claims 16-19 and, optionally, a suitable carrier, diluent or adjuvant.

29. The method according to claim 28, wherein the recombinant raccoon poxvirus vector is live and replicable.

30. The method of claim 26, wherein the immunologically effective amount of the vaccine ranges from about 4.5 Log₁₀TCID₅₀/ml to about 7.5 Log₁₀TCID₅₀/ml.

31. The method of claim 26, wherein the vaccine is administered as a single dose or as repeated doses.

32. The vaccine of claim 21, wherein the vaccine is adjuvant-free.


SEQUENCE LISTING (12 sequences)

SEQ ID NO: 1 - DNA, 6872 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgacgcggccgcctgcagaagcttggtaccatttatagcatagaaaaaaacaaaatgaaattctactatatttttacatacatatattctaaatatgaaagtggtgattgtgactagcgtagcatcgcttctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 2 - DNA, 5508 bp, Artificial
  plasmid
gagctcataaatagtaaaccgatagtgtataaagattgtgcaatgcttttgcgatcaataaatggatcacaaccagtatctgttgacgatgttcttcgcagatgatgattcattttttaaatatttagctagtcaagacgatgaatcttcattatccgatatattgcaaattacccaatatcttgattttttattgttgttattgatacagtcaaaaaataaactagaggctgtaggtcattgttatgaatctctttcggaggaatatagacaattggcgaaatttacagacactcaagagtttaaaaaactatttaataaggttcctattgctacagacggacgcgttaaacttaataaagggtatttatttgactttgttgtcagtatgatgagatttaaaaaagagtcatctataataccaaacatagatccggttcgatacatagatcctcgtcgagatatagtattttctaacgtaatggatatattaaagtctaataaagtgaacaataattaaatttttattgtcacctaggaattcgtcgacgcggccgcctgcagaagcttggtaccatttatattccaaaaaaaaaaaataaaatttcaatttttacatacatatattctaaatatgaaagtggtgattgtgactagcgtagcatcgcttctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatccggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggcttttcgctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaggatccggaaatggtagtaaaactgacagcggtatgtatgaaatgctttaaagaggcgtcgttttctaaacgtttgggaacggaaaccgagatcgaaataattggtggtgaagatatgtatcaatccgtatgcagaaagtgttacatcaacgaatgataatttttctataaaaaactaaaaataaacattgattaaattttaatataatacttaaaaatggatgttgtgtcactggataaaccgtttatgtattttgaggaaatagataatgaactagaatacgaaccagaaagtgcaaatgaggttgctaaaaaacttccatatcagggacaattaaaactattactaggagaattgttttttcttagtaagctacagagacacggtatattggacggtgccactgtagtgtatataggatcggctcctggtacacacatacgttatctacgtgatcatttctataatttaggggtaatcatcaaatggatgctaattgacgggcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - DNA, 6820 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgacgcggccgcctgcagaagcttggtaccatgggtatttatattccaaaaaaaaaaaataaaatttcaatttttgctctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 4 - DNA, 8373 bp, Artificial
  plasmid
ttattggacactagataatcatcacatgttaccacaaaattatataatgtataaatgcgaaattattaaacgcaaatatccatgggaaaacgcgcagtatacagacgattttttacagtatttggagagttttataggaagtatatagagtagaaccagaattttgtaaaaataaatcacatttttatactaatatgaaacaactatcgatagttatattgctactatcgatagtatatacaaccaaacctcatcctacacagatatcaaaaaaactaggcgatgatgctactctatcgtgtaatagaaacaatacacatggatatcttgtcatgagttcttggtataagaaaccagactccattattctcttagcagccaaaaacgatgtcgtatactttgatgattatacagcggataaagtatcatacgattcaccgtatgatactctagctacaattattacaattaaatcattgacatctggagatgcaggtacttatatatgcgcattctttataacatcaacaaatgatacggataaaatagattatgaagaattcgtcgactcacagtccggtctcacccccgctcttgtatgattcccatgaagatatgatcttcccgctttggggagtgactgacacctccctccctgtccctctgagattgtgttgtgtaggttccgatcgattgactcttctccagcatgtcatcaggaaaattatcaacatcaaggcagtcagggcccctgcactcagtaatacatacttcccccagttcgggagacccaagtcaactcctgagatccgttcgtgcacatcgggaaggtgaacttcaacaaaatcctcagcctcgtcaccgttcttgaaaacggtagacgggtctgccagggggtgcataagggggataaccgaggataccaacaactccatatgttgctggaggagggatgattgcatctctgggattaagacattgccgtcaggtcctaatattataccattgaaaaataccccgtttacatgaggatgacacctccccccaactcttaaacacccttttgaagggatgatctcattccaagttctgactgacttgtagtgagcatcggcttccatcaaggtcttgttgaatatggtatatgcttttccaaacccagggacaagttttcttaaatgactgagacgtctgaaactcactgacttggtggtcatgatggactctagtgcatccagacactcctctctcttcttgaccaactcctctacaacaaggtgctcaatttcgtctgagcgaaagtcgtgcaaattgatcaactgaccgggagggcaccatttggtttcatttgatgtttgcatcgagacccatgttccatccataagtctaagtccgagaactccacataactggagtttgcatgctccttttaaagacttatataggcctctttcatctacaaagccgcaagtctcactccctttggatgctctcttccctctactattggtaaaaatgtcacaagacatccctagtctcggattctcgggcatccaaatggtgtaatcgtggttagtggagcagtaggtagaagacaccgctactcctgagcacttcccgccagggaagaccctcgagtgaagggatctgtcatatgggtccaaatctgccacacttggagatatgataacgagagactccttggtggtttttacagttcgaagccagtggtagtcagggtacggattgtgtagagactcttcatatctggggtcaccggccatcttccagttgtacgcggctctacatgcatctggtgttgggcggaaatgctttcttttgaacgtggttgtgacataaccaacgaagttagtgtaggtttcagcctccgtcacaacgcctgtgcaagtgaacccgttcatttttatggctgagatgtatccaactttaagttccatgtaggagaaccctgacaggttggtgcatccttcgtcctccactaccaaattgtttgggcagctgaggtgatgtatgtcaatcgggctccagggaccaagcttgtctggtatcgtgtaaatagggaatttcccaaaacacaatggaaaaaccagaaggggtacaaacaggagagcctgaggaaccggtaccatgggtatttatattccaaaaaaaaaaaataaaatttcaatttttgctctagacatctatatactatatagtaataccaatactcaagactacgaaactgatacaatctcttatcatgtgggtaatgttctcgatgtcgatagccatatgcccggtagttgcgatatacataaactgatcactaattccaaacccacccgctttttatagtaagtttttcacccataaataataaatacaataattaatttctcgtaaaagtagaaaatatattctaatttattgcacggtaaggaagtagaatcataaagaacagtgacatggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctcaaggtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatcaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccggtcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccgttcgctaccattaccagttggtctggtgtcaaaaataaggatcctcgataccaacaacggtagaaagtgttacaatatctactacaaaatatacaactagtgactttatagagatatttggcattgtttcactaattttattattggccgtggcgattttctgtattatatattatttctgtagtggacggtctcgtaaacaagaaacaaatatattatagattttaactcagataaatgtctggaataattaaatctatcgttttgagcggaccatctggttccggcaagacagctatagtcaggagactcttacaagattatggaaatatatttggatttgtggtatcccataccactagatttcctcgtcctatggaacgagaaggtgtcgtctaccattacgttaacagagaggccatttggaagggaatagccgctggaaacttgctagaacatacagagtttttgggaaatatttatgggacttctaaaacatccatgaacacagctgctattaataatcgtatatgtgttatggatttaaacattgacggagttaggagtcttaaaaacacatacttgatgccttactctgtttatataagacctacatctcttaaaatggtagaaactgcatgccctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagagctc

SEQ ID NO: 5 - DNA, 702 bp, Porcine circovirus
  misc_feature (1)..(702): Porcine circovirus type 2A ORF2
atgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcagaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccaccattgattacttccaaccaaataacaaaaggaatcagctttggatgaggctacaaacctctagaaatgtggaccacgtaggcctcggcactgcgttcgaaaacagtatatacgaccaggactacaatatccgtgtaaccatgtatgtacaattcagagaatttaatcttaaagaccccccacttaaaccctaa

SEQ ID NO: 6 - DNA, 702 bp, Porcine circovirus
  misc_feature (1)..(702): Porcine circovirus type 2B ORF2
atgacgtatccaaggaggcgttaccgaagaagaagacaccgcccccgcagccatcttggccagatcctccgccgccgcccctggctcgtccacccccgccaccgttaccgctggagaaggaaaaatggcatcttcaacacccgcctctcccgcaccttcggatatactgtcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccggtactttacccccaaacctgtcctagattccactattgattacttccaaccaaacaacaaaagaaaccagctgtggctgagactacaaactgctggaaatgtagaccacgtaggcctcggcactgcgttcgaaaacagtatatacgaccaggaatacaatatccgtgtaaccatgtatgtacaattcagagaatttaattttaaagaccccccacttaacccttaa

SEQ ID NO: 7 - DNA, 765 bp, Porcine reproductive and respiratory syndrome virus
  misc_feature (1)..(765): Porcine Reproductive and Respiratory Syndrome Virus strain ISU12 ORF3
atggctaatagctgtacattcctccatattttcctctgttgcagcttcttgtactctttttgttgtgctgtggttgcgggttccaatgctacgtactgtttttggtttccgctggttaggggcaatttttctttcgaactcacggtgaactacacggtgtgcccgccttgcctcacccggcaagcagccgcagaggcctacgaacccggtaggtccctttggtgcaggatagggcatgatcgatgtggggaggacgatcatgatgaactagggtttgtggtgccgcctggcctctccagcgaaggccacttgaccagtgcttacgcctggttggcgttcctgtccttcagctatacggcccagttccatcccgagatattcgggatagggaatgtgagtcgagtctatgttgacatcaagcaccaattcatttgcgctgttcatgatgggcagaacaccaccttgccccaccatgacaacatttcagccgtgtttcagacctattaccagcatcaggtcgacggcggcaattggtttcacctagaatggctgcgtcccttcttttcctcttggttggttttaaatgtctcttggtttctcaggcgttcgcctgcaagccatgtttcagttcgagtctttcagacatcaagaccaacaccaccgcagcggcaggctttgctgtcctccaagacatcagttgccttaggcatcgcaactcggcctctgaggcgattcgcaaagtccctcagtgccgcacggcgatag

SEQ ID NO: 8 - DNA, 537 bp, Porcine reproductive and respiratory syndrome virus
  misc_feature (1)..(537): Porcine Reproductive and Respiratory Syndrome Virus strain ISU12 ORF4
atggctgcgtcccttcttttcctcttggttggttttaaatgtctcttggtttctcaggcgttcgcctgcaagccatgtttcagttcgagtctttcagacatcaagaccaacaccaccgcagcggcaggctttgctgtcctccaagacatcagttgccttaggcatcgcaactcggcctctgaggcgattcgcaaagtccctcagtgccgcacggcgatagggacacccgtgtatatcactgtcacagccaatgttaccgatgagaattatttgcattcctctgatcttctcatgctttcttcttgccttttctatgcttctgagatgagtgaaaagggatttaaggtggtatttggcaatgtgtcaggcatcgtggcagtgtgcgtcaacttcaccagttacgtccaacatgtcaaggaatttacccaacgttccttggtagttgaccatgtgcggctgctccatttcatgacgcccgagaccatgaggtgggcaactgttttagcctgtctttttgccattctgttggcaatttga

SEQ ID NO: 9 - DNA, 603 bp, Porcine reproductive and respiratory syndrome virus
  misc_feature (1)..(603): Porcine Reproductive and Respiratory Syndrome Virus strain ISU12 ORF5
atgttggggaaatgcttgaccgcgggctgttgctcgcaattgctttttttgtggtgtatcgtgccgtcttgttttgttgcgctcgtcagcgccaacgggaacagcagctcaaatttacagctgatttacaacttgacgctatgtgagctgaatggcacagattggctagctaataaatttgactgggcagtggagtgttttgtcatttttcctgtgttgactcacattgtctcttatggtgccctcactactagccatttccttgacacagtcggtctggtcactgtgtctaccgctgggtttgttcacgggcggtatgttctgagtagcatctacgcggtctgtgccctggctgcgttgatttgcttcgtcattaggcttgcgaagaattgcatgtcctggcgctactcatgtaccagatataccaactttcttctggacactaagggcagactctatcgttggcggtcgcctgtcatcatagagaaaaggggcaaagttgaggtcgaaggtcacctgatcgacctcaaaagagttgtgcttgatggttccgcggctacccctgtaaccagagtttcagcggaacaatggggtcgtccttag

SEQ ID NO: 10 - DNA, 525 bp, Porcine reproductive and respiratory syndrome virus
  misc_feature (1)..(525): Porcine Reproductive and Respiratory Syndrome Virus starin ISU12 ORF6
atggggtcgtccttagatgacttctgtcatgatagcacggctccacaaaaggtgctcttggcgttttctattacctacacgccagtgatgatatatgccctaaaggtgagtcgcggccgactgctagggcttctgcaccttttgatcttcctgaattgtgctttcaccttcgggtacatgacattcgtgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataa

SEQ ID NO: 11 - DNA, 372 bp, Porcine reproductive and respiratory syndrome virus
  misc_feature (1)..(372): Porcine Reproductive and Respiratory Syndrome Virus strain ISU12 ORF7
atgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccatttccctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatga

SEQ ID NO: 12 - DNA, 606 bp, Porcine reproductive and respiratory syndrome virus
  misc_feature (1)..(606): Porcine Reproductive and Respiratory Syndrome Virus strain Olot91 ORF5
atgagatgttctcacaaattggggcgtttcttgactcctcactcttgcttctggtggctttttttgctgtgtaccggcttgtcctggtcctttgtcgctggcagcggcagcagctcgacataccaatacatatataacttaacgatatgcgagctgaatgggaccgactggttgtccaaccattttgattgggcagtcgagacctttgtgctttacccggttgccactcatatcctctcactgggttttctcacaacaagccatttttttgacgcgctcggtctcggcgctgtgtccactacaggatttgttggcgggcggtatgtactcagcagcgtgtacggcgcttgtgctttcgcagcgctcgtatgttttgtcatccgtgctgttaaaaattgcatggcttgccgctatgcccacacccggtttaccaacttcattgtggacgaccgggggagaatccatcggtggaagtctccaatagtggtagagaaattgggcaaagctgaagtcggtggcgaccttgtcaccatcaaacatgtcgtcctcgaaggggttaaagctcaacccttgacgaggacttcggctgagcaatgggaagcctag